(19) United States
(12) Patent Application Publication
(10) Pub. No.: US 2023/0390305 A1
(43) Pub. Date: Dec. 7, 2023

(54) AMORPHOUS SOLID DISPERSION GANAXOLONE FORMULATION

(71) Applicant: MARINUS PHARMACEUTICALS, INC., Radnor, PA (US)

(72) Inventors: David CZEKAI, Radnor, PA (US); Vinod TULIANI, Radnor, PA (US); Michael SAPORITO, Radnor, PA (US)

(21) Appl. No.: 18/355,684

(22) Filed: Jul. 20, 2023

Related U.S. Application Data
(63) Continuation of application No. PCT/US2022/077514, filed on Oct. 4, 2022.
(60) Provisional application No. 63/396,665, filed on Aug. 10, 2022.
(60) Provisional application No. 63/252,098, filed on Oct. 4, 2021.

(51) Int. Cl. A61K 31/57; A61K 9/14; A61K 9/10; A61K 47/32

(52) U.S. Cl. CPC A61K 31/57; A61K 9/146; A61K 9/10; A61K 47/32

(57) ABSTRACT

This disclosure relates to a solid pharmaceutical formulation comprising an amorphous neurosteroid dispersed in a polymer matrix that is suitable for oral administration. The disclosure also relates to methods for effectively treating an epileptic disorder, central nervous system disorder, or a neurological disorder. The methods disclosed herein comprise orally administering to a subject in need thereof a therapeutically effective amount of the solid pharmaceutical formulation disclosed herein comprising an amorphous neurosteroid, preferably ganaxolone dispersed in a polymer matrix.

### 1. RELATED APPLICATIONS

This application claims the benefit of U.S. Provisional Patent Application No. 63/252,098, filed Oct. 4, 2021 and U.S. Provisional Patent Application No. 63/396,665, filed Aug. 10, 2022, the entire contents of each of which are incorporated herein by reference.

### 2. BACKGROUND

Neurosteroids modulate neuron activity via interactions with neurotransmitter receptors impacting neuron excitability and neurotransmitter secretion (Schumacher et al., 2000). Some neurosteroids, such as allopregnanolone and ganaxolone, are positive allosteric modulators of GABA-A receptors. This neurosteroid-mediated positive allosteric modulation of GABA-A receptors increases GABA activity and inhibits neuronal excitability. As exogenously administered pharmacological agents, inhibitory neurosteroids, such as allopregnanolone and ganaxolone, have anticonvulsant activity. Aside from anticonvulsant actions, inhibitory neurosteroids may be useful as anxiolytic, hypotonic, and anesthetic agents. However, neurosteroids, such as ganaxolone, have very low aqueous solubility, which has limited their clinical use particularly with respect to oral dosage forms. Related to the very low solubility of neurosteroids, such as ganaxolone and allopregnanolone, it has been difficult to develop oral dosage forms of these molecules that are both effective (e.g., provide sufficient bioavailability, absorption and exposure (AUC)) and safe.

Oral dosage forms of neurosteroids are desirable for certain patient populations, particularly pediatric patients. Attempts to develop oral ganaxolone have been made. For example, it took Marinus Pharmaceuticals, Inc. more than 10 years and many formulations to develop an oral nanoparticulate suspension of ganaxolone for oral administration. Other attempts to develop oral neurosteroid formulations have largely been unsuccessful. For example, a clinical trial using orally-active allopregnanolone, SAGE-217, failed for non-compliance issues, with about 10% of patients presenting no blood drug levels (Pinna, (2020), Front. Endocrinol., 11:236).

Based upon the difficulties in developing suitable oral neurosteroid formulations, there exists in the art a need for improved neurosteroid formulations suitable for oral administration.

### 3. SUMMARY

This disclosure relates to solid pharmaceutical formulations comprising an amorphous neurosteroid, such as ganaxolone that is dispersed in a polymer matrix. Ganaxolone has been suggested and studied as a therapy for particular seizure and epilepsy-related disorders, central nervous system disorders and neurological disorders, such as depression. However, the results of such investigations have been variable. As noted above, ganaxolone and other neurosteroids are typically poorly soluble or essentially in soluble drugs that do not generally provide suitable bioavailability properties upon oral administration.

The inventors have developed new solid pharmaceutical neurosteroid formulations that are suitable for oral administration. The formulations surprisingly and unexpectedly have enhanced pharmacokinetic (PK) and pharmacodynamic (PD) profile as compared to oral ganaxolone nanosuspension formulation described in the prior art (see, International Application No. PCT/US2006/045626). As described and exemplified herein, the disclosed formulations have improved oral bioavailability of the neurosteroid after oral administration to the subject, such as a human. Without being bound by theory, it is believed that the increased bioavailability increases exposure to the neurosteroid (e.g., ganaxolone) and/or makes it easier to maintain therapeutic serum concentration of the neurosteroid (e.g., ganaxolone).

The formulations described herein, can be administered to achieve effective serum concentrations of neurosteroid using a lower dose of neurosteroid, in comparison to prior oral formulations, which is expected to also decrease risk of undesired side effects.

Accordingly, this disclosure relates to novel solid pharmaceutical formulations comprising an amorphous neurosteroid, preferably ganaxolone dispersed in a polymer matrix. These solid pharmaceutical formulations are suitable for oral administration. The disclosure also relates to methods for treating an epileptic disorder or a central nervous system disorder. The methods disclosed herein comprise orally administering to a subject in need thereof a therapeutically effective amount of the solid pharmaceutical formulations disclosed herein comprising an amorphous neurosteroid, preferably ganaxolone, dispersed in a polymer matrix.

The pharmaceutical formulations disclosed herein comprise a neurosteroid dispersed in a polymer matrix. The neurosteroid is amorphous and is of Formula I:

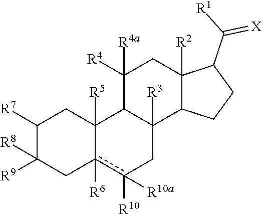

or a pharmaceutically acceptable salt thereof, wherein:

[symbol] is a double or a single bond;

X is 0, S, or NR¹¹;

R¹ is hydrogen, hydroxyl, optionally substituted alkyl, optionally substituted heteroalkyl, optionally substituted heteroaryl, optionally substituted heteroarylalkyl, optionally substituted aryl, or optionally substituted arylalkyl;

R⁴ is hydrogen, optionally substituted alkyl, optionally substituted cycloalkyl, or optionally substituted (cycloalkyl)alkyl, or —OR⁴⁰, where R⁴⁰ is hydrogen, optionally substituted alkyl, optionally substituted cycloalkyl, or optionally substituted (cycloalkyl)alkyl, or optionally substituted C₃-C₆ carbocycle;

R^{4a} is hydrogen or R⁴ or R^{4a} are taken together to form an oxo (═O) group;

R², R^{3′} R⁵ and R⁶, are each independently hydrogen, hydroxyl, halogen, optionally substituted alkyl, optionally substituted cycloalkyl, or optionally substituted (cycloalkyl)alkyl, or optionally substituted heteroalkyl;

R⁷ is hydrogen, halogen, optionally substituted alkyl, optionally substituted C₃-C₆ carbocycle, optionally substituted (C₃-C₆ carbocycle)alkyl or —OR⁷⁰ where R⁷ is hydrogen, optionally substituted alkyl, optionally substituted C₃-C₆ carbocycle, or optionally substituted (C₃-C₆ carbocycle)alkyl;

R⁸ is hydrogen, optionally substituted alkyl or optionally substituted C₃-

C₆ carbocycle, and R⁹ is hydroxyl; or

R⁸ and R⁹ are taken together to form an oxo group;

R¹⁰ is hydrogen, halogen, hydroxyl, optionally substituted alkyl, optionally substituted heteroalkyl, optionally substituted C₃-C₆ carbocyle, or optionally substituted (C₃-C₆ carbocycle)alkyl, [symbol] and R¹⁰ is hydrogen, halogen, or optionally substituted alkyl, provided that if is a double bond R¹⁰ is absent;

each alkyl is a C₁-C₁₀ alkyl and optionally contains one or more single bonds replaced by a double or triple bond; and

each heteroalkyl group is an alkyl group in which one or more methyl group is replaced by an independently chosen —O—, —S—, —N(R¹¹)—, —S(═O)— or —S(═O)2-, where R¹¹ is independently chosen at each occurrence and is hydrogen, alkyl, or alkyl in which one or more methylene group is replaced by —O—, —S—, —H, or —N-alkyl.

The neurosteroid can be ganaxolone, allopregnanolone, alphaxolone, alphadolone, hydroxydione, minaxolone, pregnanolone, acebrochol, isopregnanolone, or tetrahydrocorticosterone or a pharmaceutically acceptable salt thereof. Preferably, the neurosteroid is ganaxolone.

The polymer matrix can comprise a neutral water-soluble polymer and optionally an amphiphilic polymer.

The solid pharmaceutical formulation can comprise ganaxolone in an amount of about 10% to about 40% by weight, the polymer can comprise a neutral water-soluble polymer in an amount of about 50% to about 90% by weight, and can optionally comprise an amphiphilic polymer in an amount of about 1% to about 30% by weight. Ganaxolone is amorphous and all weight percentages are with respect to the solid pharmaceutical formulation.

The neutral water-soluble polymer can comprise polyvinyl pyrrolidone, or a polyvinyl pyrrolidone co-polymer. The polyvinyl pyrrolidone co-polymer can be a polymer that comprises N-vinyl pyrrolodoneand vinyl acetate repeat units. The polyvinylpyrrolidone co-polymer can be a random co-polymer, a block co-polymer or a graft-co-polymer.

The polyvinyl pyrrolidone copolymer can be of Formula II:

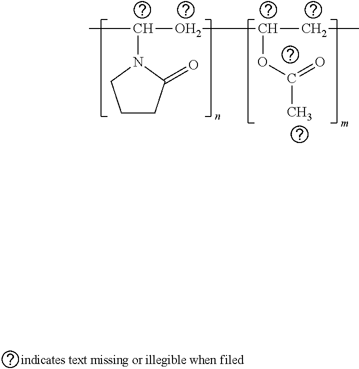

The water-soluble polymer can have a weight average molecular weight of about 30,000 to about 150,000. The water-soluble polymer can have a weight average molecular weight of about 45,000 to about 70,000.

The water-soluble copolymer can be Kollidon V64 (BASF, copovidone, copolyvidone, vinylpyrrolidone-vinyl acetate copolymer).

The solid pharmaceutical formulation can further comprise the optional amphiphilic polymer. The amphiphilic polymer is a polyvinyl caprolactam-polyvinyl acetate-polyethylene glycol graft co-polymer. The amphiphilic polymer can have Formula III:

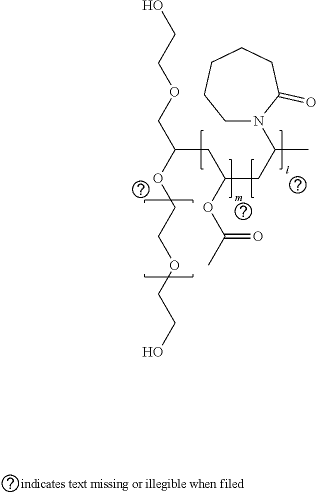

The amorphous neurosteroid can be present in an amount of about 10% to about 40% by weight. The amorphous neurosteroid can be present in an amount of about 20% to about 30% by weight. The water-soluble polymer can present in an amount of about 50% to about 90% by weight. The water-soluble polymer can be present in an amount of about 50% to about 80% by weight. The optional amphiphilic polymer can be present in an amount of about 1% to about 30% by weight. The optional amphiphilic polymer can be present in an amount of about 20% to about 30% by weight.

The amorphous neurosteroid can have less than about 30% crystallinity. The amorphous neurosteroid can have less than about 20% crystallinity. The amorphous neurosteroid can have less than about 10% crystallinity. The amorphous neurosteroid can have less than about 5% crystallinity. The amorphous ganaxolone can have less than about 30% crystallinity. The amorphous ganaxolone can have less than about 20% crystallinity. The amorphous ganaxolone can have less than about 10% crystallinity. The amorphous ganaxolone can have less than about 5% crystallinity. The crystallinity is generally measured using powder X-ray diffraction.

The w/w ratio of copolymer to ganaxolone can be about 50:10 to about 90:40. The w/w ratio of copolymer to amphiphilic polymer to ganaxolone is about 50:1:10 to about 90:30:40. The ratio of copolymer to ganaxolone can be about 70:20 or higher. The ratio of copolymer to ganaxolone can be about 60:20 to about 90:40. The ratio of copolymer to amphiphilic polymer to ganaxolone can be about 50:20:20 or higher.

The solid pharmaceutical formulation can be in the form of a powder. The powder can comprises particles that have a volume weighted median diameter from about 5 nm to about 1000 nm. The solid pharmaceutical formulation disclosed herein can comprise a capsule or tablet.

The disclosure also relates to methods for treating a seizure disorder, an epilepsy disorder, a genetic epilepsy disorder, a epilepsy related disorder, a central nervous system disorder, a neurological disorder, or neurodegenerative disorders comprising administering to a subject in need thereof a therapeutically effective amount of the solid pharmaceutical formulation disclosed herein.

The solid pharmaceutical formulation can be administered one time per day. The solid pharmaceutical formulation can be administered two times per day. The solid pharmaceutical formulation can be administered three times per day. The solid pharmaceutical formulation can be administered orally.

The solid pharmaceutical formulation can be administered in an amount up to from about 1 mg/kg to about 200 mg/kg. The solid pharmaceutical formulation can be administered in an amount at about 10 mg/kg, 20 mg/kg, or 50 mg/kg.

The epilepsy disorder can be a focal seizure, a generalized seizure, progressive myoclonic epilepsy, reflex epilepsy, Landau-Kleffner Syndrome, Ohtahara syndrome, Rasmussen's syndrome, infantile spasms (or West syndrome), Lennox-Gastaut syndrome (LGS), Rett syndrome, Dravet syndrome, Doose syndrome, CDKL5 disorder, intractable childhood epilepsy (ICE), childhood absence epilepsy (CAE), juvenile myoclonic epilepsy (JME), essential tremor, acute repetitive seizures, benign rolandic epilepsy, status epilepticus, refractory status epilepticus, super-refractory status epilepticus, PCDH19 pediatric epilepsy, increased seizure activity, a breakthrough seizures or an infantile spasms.

The neurological disorder can be anxiety, agitation, or depression

The solid pharmaceutical formulation can be administered in an amount effective to produce a maximum ganaxolone plasma concentration (Cₘₐ) of about 100 ng/ml to about 1200 ng/ml. The solid pharmaceutical formulation can be administered in an amount effective to produce an area under the curve (AUC₀₋₈ₕᵣₛ) from about 100 ng/ml/hr to about 1200 ng/ml/hr. The solid pharmaceutical formulation can be administered in an amount effective to produce an average ganaxolone plasma concentration (Cₐᵥₑ) of about 100 ng/ml to about 1,200 ng/ml. The solid pharmaceutical formulation can be administered in an amount effective to produce an minimum ganaxolone plasma concentration (Cₘᵢₙ) of about 100 ng/ml to about 1,200 ng/ml.

### 4. BRIEF DESCRIPTION OF THE DRAWINGS

FIG. 1A-1B are graphs comparing ganaxolone suspension (GNX Suspension) and amorphous ganaxolone hot melt extruded formulations (HME-1: 30% ganaxolone 70% Kollidon VA64 by weight; HME-2: 30% ganaxolone 50% Kollidon VA64 20% Soluplus by weight). FIG. 1A shows ganaxolone plasma concentration for ganaxolone suspension and amorphous ganaxolone formulations HME-1 and HME-2 at 10 mg/kg. FIG. 1B shows ganaxolone plasma concentration for ganaxolone suspension and amorphous ganaxolone hot melt extruded formulations 1 and 2 at 20 mg/kg.

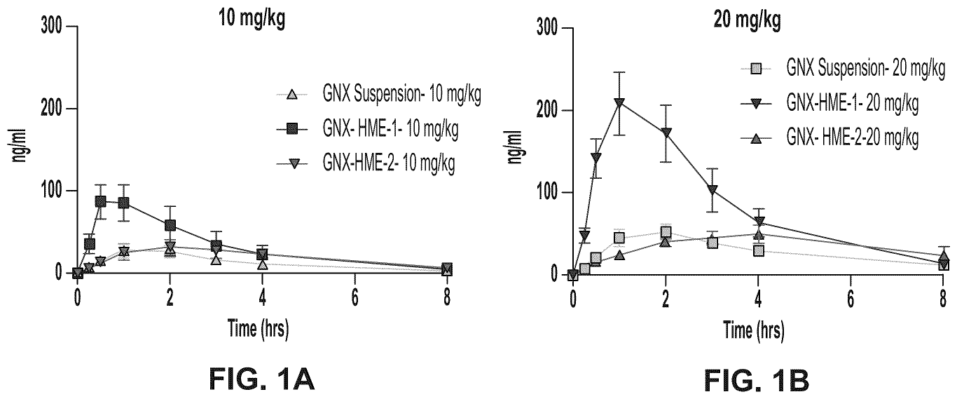

FIG. 2A-2B are graphs showing ganaxolone plasma concentrations for amorphous ganaxolone HME-1 formulation at 10 mg/kg and 20 mg/kg (FIG. 2A) and amorphous ganaxolone HME-2 formulation at 10 mg/kg and 20 mg/kg (FIG. 2B). (HME-1: 30% ganaxolone 70% Kollidon VA64 by weight; HME-2: 30% ganaxolone 50% Kollidon VA64 20% Soluplus by weight)

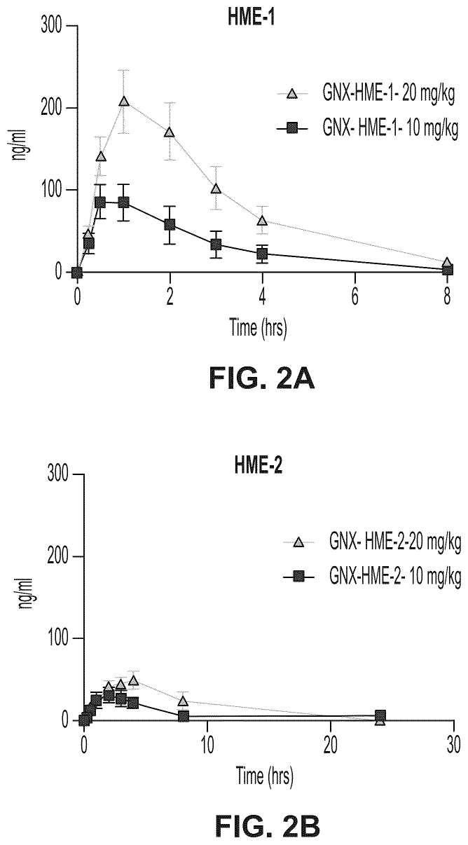

FIG. 3 is a graph showing the AUC of the ganaxolone suspension and amorphous ganaxolone HME-1 and HME-2 formulations at 10 mg/kg and 20 mg/kg. (HME-1: 30% ganaxolone 70% Kollidon VA64 by weight; HME-2: 30% ganaxolone 50% Kollidon VA64 20% Soluplus by weight)

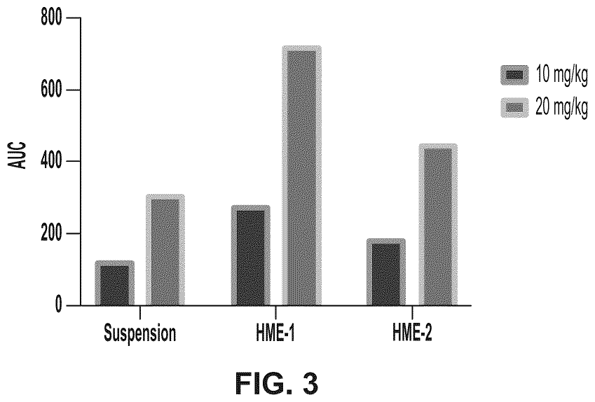

FIG. 4 is a graph showing the mean±SD concentration of the rat PK study for Groups 1-6 administered with either 10 mg/kg or 20 mg/kg of amorphous ganaxolone HME formulation.

FIG. 5 is a graph showing ganaxolone plasma concentration in subjects administered with ganaxolone suspension at 400 mg fed state and amorphous ganaxolone HME-1 (HME-1: 30% ganaxolone 70% Kollidon VA64 by weight) formulation at 100 mg, 200 mg, and 400 mg at fasted and fed states.

FIG. 6 is a graph showing ganaxolone plasma concentration in subjects administered with ganaxolone suspension at 600 mg fed state and amorphous ganaxolone HME-1 formulation at 400 mg, 600 mg, and 900 mg at fasted and/or fed state.

FIG. 7 is a graph showing AUC₀₋ₜ of ganaxolone in plasma (ng*h/ml) in subjects administered with ganaxolone suspension at 400 mg (fed) and 600 mg (fed) and HME-1 formulation at 100 mg (fasted and fed), 200 mg (fasted and fed), 400 mg (fasted or fed), 600 mg (fed), and 900 mg (fasted or fed).

FIG. 8 is a graph showing AUC₀. of ganaxolone in plasma (ng*h/ml) in subjects administered with ganaxolone suspension at 400 mg (fed) and 600 mg (fed) and HME-1 formulation at 100 mg (fasted and fed), 200 mg (fed), 400 mg (fasted or fed), 600 mg (fed), and 900 mg (fasted or fed). The graph shows that HME-1 AUC is linear with ganaxolone dose.

FIG. 9 is a graph showing AUC₀. of ganaxolone in plasma (ng*h/ml) in subjects administered with ganaxolone suspension at 400 mg (fed), 600 mg (fed), and 800 mg (fed) and HME-1 formulation at 100 mg (fasted and fed), 200 mg (fed), 400 mg (fasted or fed), 600 mg (fed), and 900 mg (fasted or fed). The graph shows that HME-1 AUC is linear with ganaxolone dose, whereas ganaxolone suspension plateaus between 600 and 800 mg.

### 5. DETAILED DESCRIPTION

Ganaxolone and other neurosteroids have low aqueous solubility and in many cases are essentially insoluble. Consequently, neurosteroids, such as ganaxolone, have poor bioavailability upon oral administration.

This disclosure relates to solid pharmaceutical formulation suitable for oral administration comprising a neurosteroid, such as ganaxolone that is amorphous and dispersed in a polymer matrix. In embodiments, the formulations surprisingly and unexpectedly have enhanced pharmacokinetic (PK) and pharmacodynamic (PD) profile as compared to oral ganaxolone nanosuspension formulation described in the prior art (see, International Application No. PCT/US2006/045626). The disclosure also relates to methods for effectively treating an epilepsy disorder, an epilepsy related disorder, a central nervous system disorder, or a neurological disorder. The methods disclosed herein comprise orally administering to a subject in need thereof a therapeutically effective amount of solid pharmaceutical formulations disclosed herein comprising a neurosteroid, preferably ganaxolone that is amorphous and dispersed in a polymer matrix.

The solid pharmaceutical formulations disclosed herein comprises a neurosteroid that is amorphous and dispersed in a polymer matrix. Ganaxolone is a preferred neurosteroid. The polymer matrix comprises a neutral water-soluble polymer and optionally an amphiphilic polymer.

Additional description of the solid pharmaceutical formulations containing a neurosteroid in a polymer matrix and guidance for the practice of the formulations are provided herein. For ease of presentation, further details and guidance are provided with respect to a preferred aspect of a solid pharmaceutical formulation comprising ganaxolone. It is intended that further details and guidance also relate to other neurosteroids.

A. Neurosteroids

This disclosure relates to solid pharmaceutical formulations that are suitable for oral administration. Neurosteroids, also known as neuroactive steroids, are endogenous or exogenous steroids that rapidly alter neuronal excitability through interaction with ligand-gated ion channels and other cell surface receptors. Paul et al., FASEB J. 6 (6): 2311-2322 (1992); and Lan et al., Horm Behav, 28 (4):537-544 (1994). Neurosteroids are synthesized by an endocrine gland that then reach the brain through the bloodstream and have effects on brain function. Srivastava et al., J. Neurosci. 31 (45):16056-16063 (2011). In addition to their actions on neuronal membrane receptors, some of these neurosteroids may also exert effects on gene expression via nuclear steroid hormone receptors. Neurosteroids have a wide range of potential clinical applications from sedation to treatment of epilepsy and traumatic brain injury. Reddy et al., Neurotherapeutics 6 (2):392-401 (2009); Morrow A., Pharmacol. Ther. 116 (1):1-6 (2007); and Dubrovsky B., Prog. Neuropsychopharmacol. Biol. Psychiatry 29 (2):169-192 (2005). Of particular interest are progestational hormones, also known as “progestins” or “progestogens.” Members of this family include steroid hormones disclosed in Remington's Pharmaceutical Sciences, Gennaro et al., Mack Publishing Co. (18th ed. 1990), 990-993.

Neurosteroids suitable for the solid pharmaceutical formulations described herein can have the general structural formula of pregnane, androstane, 19-norandrostanes, and norpregnane with further moieties. The neurosteroids suitable for the solid pharmaceutical formulations described herein can have a structural formula as defined by Formula I (below) or a pharmaceutically acceptable salt thereof.

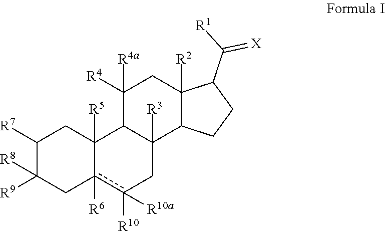

[symbol] is a double or a single bond;

X is 0, S, or NR¹¹;

R¹ is hydrogen, hydroxyl, optionally substituted alkyl, optionally substituted heteroalkyl, optionally substituted heteroaryl, optionally substituted heteroarylalkyl, optionally substituted aryl, or optionally substituted arylalkyl;

R⁴ is hydrogen, optionally substituted alkyl, optionally substituted cycloalkyl, or optionally substituted (cycloalkyl)alkyl, or —OR⁴⁰, where R⁴⁰ is hydrogen, optionally substituted alkyl, optionally substituted cycloalkyl, or optionally substituted (cycloalkyl)alkyl, or optionally substituted C₃-C₆ carbocycle;

R^{4a} is hydrogen or R⁴ or R^{4a} are taken together to form an oxo (═O) group;

R², R³, R^{5′} and R⁶, are each independently hydrogen, hydroxyl, halogen, optionally substituted alkyl, optionally substituted cycloalkyl, or optionally substituted (cycloalkyl)alkyl, or optionally substituted heteroalkyl;

R⁷ is hydrogen, halogen, optionally substituted alkyl, optionally substituted C₃-C₆ carbocycle, optionally substituted (C₃-C₆ carbocycle)alkyl or —OR⁷⁰ where R⁷ is hydrogen, optionally substituted alkyl, optionally substituted C₃-C₆ carbocycle, or optionally substituted (C₃-C₆ carbocycle)alkyl;

R⁸ is hydrogen, optionally substituted alkyl or optionally substituted C₃-C₆ carbocycle, and R⁹ is hydroxyl; or

R and R⁹ are taken together to form an oxo group;

R¹ is hydrogen, halogen, hydroxyl, optionally substituted alkyl, optionally substituted heteroalkyl, optionally substituted C₃-C₆ carbocyle, or optionally substituted (C₃-C₆ carbocycle)alkyl, and R^{1′} is hydrogen, halogen, or optionally substituted alkyl, provided that if [symbol] is a double bond R^{1′} is absent;

each alkyl is a C₁-C₁₀ alkyl and optionally contains one or more single bonds replaced by a double or triple bond; and

each heteroalkyl group is an alkyl group in which one or more methyl group is replaced by an independently chosen —O—, —S—, —N(R¹¹)—, —S(═O)— or —S(═O)2-, where R¹¹ is independently chosen at each occurrence and is hydrogen, alkyl, or alkyl in which one or more methylene group is replaced by —O—, —S—, —H, or —N-alkyl.

Compounds of Formula I include ganaxolone, allopregnanolone, alphaxolone, alphaxalone, alphadolone, hydroxydione, minaxolone, pregnanolone, acebrochol, isopregnanolone, or tetrahydrocorticosterone, their analogs, salts, and prodrugs.

Exemplary neurosteroids that may be suitable for use in the formulations disclosed herein include, but not limited to, allopregnanolone and tetrahydrodeoxycorticosterone, allotetrahydro-deoxycorticosterone, ganaxolone, alphaxolone, alphadolone, androstanediol (5α-androstan-3α,17β-diol), androsterone (5α-androstan-3α-ol-17-one), etiacholanone (5β-androstan-3α-ol-17-one), hydroxydione, minaxolone, and Althesin, allotetrahydrodeoxycorticosterone (3α,21-dihydroxy5α-pregnan-20-one; THDOC), 3 α21-dihydroxy-5β-pregnan-20-one, pregnanolone (3α-hydroxy-5p-pregnan-20-one), ganaxolone (INN, also known as CCD-1042; IUPAC name (3α,5α)-3α-hydroxy-3p-methyl-pregnan-20-one; 1-[(3R, 5S,8R,9S,1OS, 13S, 14S,17S)-3-hydroxy-3, 10, 13-trimethyl 1,2,4,5,6,7,8,9,11,12,14,15,16,17-tetradecahydrocyclopenta [a]phenanthren-17-yl]ethanone), alphaxolone, alphadolone, hydroxydione, minaxolone, and Althesin (a mixture of alphaxolone, alphadolone, tetrahydrodeoxycorticosterone, pregnenolone, dehydroepiandrosterone (DHEA), 7-substituted benz[e]indene-3-carbonitriles (see, e.g., Hu, et al., J Med. Chem., 36 (24):3956-67 (1993)); 7-(2-hydroxyethyl) benz[e]indene analogues (see, e.g., Han, et al., J Med. Chem. 38 (22):4548-56 (1995)); 3alpha-hydroxy-5alpha-pregnan20-one and 3alpha-hydroxy-5beta-pregnan-20-one analogues (see, e.g., Han, et al., J Med. Chem. 39 (21): 4218-32 (1996)); enantiomers of dehydroepiandrosterone sulfate, pregnenolone sulfate, and (3alpha,5beta)-3-hydroxypregnan-20-one sulfate (see, e.g., Nilsson, et al., J Med. Chem., 41 (14):2604-13 (1998)); 13,24-cyclo-18,21-dinorcholane analogues (see, e.g., Jiang, et al., J Med. Chem., 46 (25):5334-48 (2003)); N-acylated 17α-aza-D-homosteroid analogues (see, e.g., Covey, et al., J Med. Chem., 43 (17): 3201-4 (2000)); 5 beta-methyl-3-ketosteroid analogues (see, e.g., Zeng, et al., J Org. Chem., 65 (7):2264-6 (2000)); 18-norandrostan-17-one analogues (see, e.g., Jiang, et al.; J Org. Chem., 65 (11):3555-7 (2000)); (3alpha,5alpha)- and (3alpha, 5beta)-3-hydroxypregnan-20-one analogs (see, e.g., Zeng, et al., J Med. Chem., 48(8):3051-9 (2005)); benz[f]indenes (see, e.g., Scaglione, et al., J Med. Chem., 49(15):4595605 (2006)); enantiomers of androgens (see, e.g., Katona, et al., Eur J Med. Chem., 43(1):107-13 (2008)); cyclopenta[b]phenanthrenes and cyclopenta[b]anthracenes (see, e.g., Scaglione, et al., J Med. Chem. 51(5):1309-18 (2008)); 2beta-hydroxygonane derivatives (see, e.g., Wang, et al., Tetrahedron, 63(33):7977-7984 (2007)); A16-alphaxalone and corresponding 17-carbonitrile analogues (see, e.g., Bandyopadhyaya, et al., Bioorg Med Chem. Lett., 20(22):6680-4 (2010)); A(16) and A(17(20)) analogues of A(16)-alphaxalone (see, e.g., Stastna, et al., J Med. Chem. 54(11):3926-34 (2011)); neurosteroid analogs developed by CoCensys (now Purdue Neuroscience) (e.g., CCD-3693, C02-6749 (a.k.a., GMA-839 and WAY141839); neurosteroid analogs described in U.S. Pat. No. 7,781,421 and in PCT Patent Publications WO 2008/157460; WO 1993/003732; WO 1993/018053; WO 1994/027608; WO 1995/021617; WO 1996/016076; WO 1996/040043, as well as salts, hemisuccinates, nitrosylated, sulfates and derivatives thereof; neurosteroid analogs described International Published Application WO 2013/019711 and Hogenkamp et al., “Pharmacological profile of a 17β-heteroaryl2xzgxyyzyji neuroactive steroid” Psychopharmacology 231; 3517-3524 (2014).

While any neurosteroid can be suitable for the formulations described herein, ganaxolone is a preferred neurosteroid. Ganaxolone (CAS Reg. No. 38398-32-2, 3alpha-hydroxy, 3beta-methyl-5alpha-pregnan-20-one) is a synthetic steroid with anti-convulsant activity useful in treating epilepsy and other central nervous system disorders. Ganaxolone has the structural shown in Formula II (as provided below).

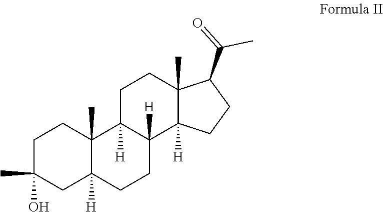

B. Solid Pharmaceutical Formulations

As described in detail herein, this disclosure relates to solid pharmaceutical formulations comprising an amorphous neurosteroid, preferably ganaxolone, that is dispersed in a polymer matrix. The solid pharmaceutical formulations comprise one or more neurosteroids. For instance, the formulation can comprise one neurosteroid or a combination of two or more neurosteroids. A preferred formulation comprises a single neurosteroid, preferably ganaxolone. The neurosteroids, such as ganaxolone, can be incorporated into the formulations as natural compounds, pharmaceutically acceptable salts and/or prodrugs or metabolites. The solid pharmaceutical formulations are suitable for oral administration.

a. Polymer Matrix

The solid pharmaceutical formulations disclosed herein comprise a neurosteroid (preferably, ganaxolone) that is dispersed in a polymer matrix. The polymer matrix can be a matrix formed by a single polymer, or preferably a mixture of polymers. A variety of polymers and copolymers can be used, typically at least one of the polymers in the matrix is water soluble. Suitable water soluble polymers include, neutral and ionic polymers and mixtures thereof. Examples of suitable neutral water-soluble polymers include, but are not limited to, hydroxyethyl cellulose, hydroxypropyl cellulose, hydroxypropyl methylcellulose, methylcellulose, polyvinyl alcohol, polyvinyl alcohol copolymers or derivatives, vinyl acetate-vinyl alcohol copolymers, polyvinylpyrrolidone, hydrolyzed polyvinylpyrrolidone, polyacrylamide, poly(methacrylamide), dextran, polyethylene glycol, and polyoxyethylene and polyoxypropylene block copolymers and the like, and mixtures thereof.

In some embodiments, that neutral water soluble polymer is hydroxyethyl cellulose (HEC), hydroxypropyl cellulose (HPC), polyvinylpyrrolidone (PVP), polyvinylpyrrolidone/vinyl acetate copolymer (PVP/VA), hydroxypropyl methylcellulose (HPMC), methylcellulose (MC), polyethylene glycol (PEG), polyvinyl alcohol (PVA), and the mixtures thereof.

Examples of anionic water-soluble polymers include, but are not limited to, sodium carboxymethylcellulose, sodium carboxymethyl hydroxyethylcellulose, pectin, carrageenan, carboxymethylguar gum, sodium alginate, anionic polyacrylamide copolymers, alkali-soluble latex, carboxymethyl methylcellulose, carboxymethyl hydroxypropyl guar, and other anionic carbohydrate derivatives, as well as mixtures including one or more of these polymers. Other suitable anionic polymers include polymaleic acid, polysulfonates, and mixtures thereof.

Examples of cationic water soluble polymers include, but are not limited to, cationic cellulose derivatives such as, for example, the quaternized hydroxyethyl cellulose obtainable from Amerchol under the name of Polymer JR 400®; cationic starch; copolymers of diallyl ammonium salts and acrylamides; quaternized vinyl pyrrolidone/vinyl imidazole polymers such as, for example, Luviquat®, (BASF); condensation products of polyglycols and amines; quaternized collagen polypeptides such as, for example, lauryldimonium hydroxypropyl hydrolyzed collagen (Lamequat® L, Grunau GmbH); quaternized wheat polypeptides; polyethyleneimine; cationic silicone polymers such as, for example, Amidomethicone; copolymers of adipic acid and dimethylaminohydroxypropyl diethylenetriamine (Cartaretine® Sandoz AG); polyacrylamide polymers and copolymers; copolymers of acrylic acid with dimethyl diallyl ammonium chloride (Merquat® 550, Chemviron); polyaminopolyamides as described, for example, in FR-A 2 252 840; epichlorohydrin/dimethylamine polymers (EPI-DMA) or epihalohydrin reaction products of polyaminoamides obtained by reaction of polyamines with dicarboxylic acids and crosslinked water-soluble polymers thereof; diallyldimethyl ammonium chloride (DADMAC) and polymers of DADMAC such as DADMAC/acrylamide copolymers; cationic chitin derivatives such as, for example, quaternized chitosan, optionally in microcrystalline distribution, condensation products of dihaloalkyls, for example dibromo butane, with bis-dialkylamines, for example bis-dimethylamino-1,3-propane, cationic guar gum such as, for example, Jaguar® CBS, Jaguar®C-17, Jaguar®(C-16 of Celanese, USA, quaternized ammonium salt polymers such as, for example, Mirapol®A-15, Mirapol®AD-1, Mirapol®AZ-1 of Miranol, USA; ionene polymers and mixtures thereof.

Preferred polymers for use in the formulations of this disclosure include KOLLIDON VA64 (BASF, copovidone, copolyvidone, poly(vinyl pyrrolidone-co-vinyl acetate)), HPMCAS-M (hypromellose acetate succinate), HPMCAS-L (hypromellose acetate succinate), HPMCAS-H (hypromellose acetate succinate), PVP K30 (polyvinylpyrrolidone), SOLUPLUS (polyvinylcaprolactam-polyvinyl acetate-polyethylene glycol graft copolymer), EUDRAGIT S100 (poly(methacylic acid-co-methyl methacrylate)), EUDRAGIT L100 poly(methacylic acid-co-methyl methacrylate), EUDRAGIT L100-55 (poly(methacylic acid-co-ethyl acrylate)), EUDRAGIT EPO (poly(butyl methacrylate-co-(2-demethylaminoeethyl) methacrylate-co-methyl methacrylate), and combinations thereof. For example, the polymer matrix can include KOLLIDON VA64, SOLUPLUS or a combination of KOLLIDON AV64 and SOLUPLUS.

Particularly preferred polymers for use in the formulations of this disclosure include homopolymers and co-polymers of N-vinyl lactams, including homopolymers and co-polymers of N-vinyl pyrrolidone, such as polyvinylpyrrolidone (PVP), and copolymers of PVP and vinyl acetate. Preferably, such polymers are neutral and water soluble. Such polymers can comprises polyvinyl pyrrolidone, or a polyvinyl pyrrolidone co-polymer. Examples of polyvinylpyrrolidone include homopolymers of vinylpyrrolidone such as N-vinyl-2-pyrrolidone and N-vinyl-4-pyrrolidone, and copolymers thereof. Suitable polymers of this type contain N-vinyl pyrrolidone monomers, such as N-vinyl-2-pyrrolidone, N-vinyl-4-pyrrolidone, and derivatives thereof. Suitable derivatives of these monomers include derivatives that include substituents on the pyrrolidone ring, such as N-vinyl-3-methylpyrrolidone, N-vinyl-5-methylpyrrolidone, N-vinyl-3,3,5-trimethylpyrrolidone, and N-vinyl-3-benzylpyrrolidone and the like. Examples of the monomers that can be used to form copolymers with an N-vinylpyrrolidone monomer include (meth) acrylic monomers such as (meth) acrylic acid, methyl (meth) acrylate, ethyl (meth) acrylate, isopropyl (meth) acrylate, unsaturated carboxylic acids such as fumaric acid, maleic acid, itaconic acid, alkylenes (e.g., ethylene, propylene) vinyl chloride, vinyl acetate, vinyl alcohol, styrene, vinyl toluene, divinylbenzene, vinylidene chloride, ethylene tetrafluoride, and vinylidene fluoride and the like.

Furthermore, a modified polyvinyl pyrrolidone resin and the like can be used for or to form the formulations disclosed herein. The modified polyvinyl pyrrolidone resin can be a copolymer of an N-vinyl pyrrolidone monomer and another monomer. The co-polymer can be a random copolymer, block copolymer, graft copolymer, and any desired combination thereof can be used in the formulations of this disclosure.

A preferred neutral water-soluble polymer is a polymer that comprises N-vinyl pyrrolodone and vinyl acetate repeat units. For example, Kollidon V64 (BASF, copovidone, copolyvidone, vinylpyrrolidone-vinyl acetate copolymer) is particularly suitable for the solid pharmaceutical formulations disclosed herein.

Preferably, the polymer matrix comprises a neutral water-soluble copolymer and optionally an amphiphilic polymer. In embodiments, the polymer matrix comprises a neutral water-soluble copolymer. In embodiments, the polymer matrix comprises a neutral water-soluble copolymer and an amphiphilic polymer. The neutral water-soluble copolymer can comprises polyvinyl pyrrolidone, or a polyvinyl pyrrolidone co-polymer. The polyvinyl pyrrolidone co-polymer can be a polymer that comprises N-vinyl pyrrolodone and vinyl acetate repeat units. The polyvinylpyrrolidone co-polymer is a random co-polymer, a block co-polymer or a graft-co-polymer.

The polyvinyl pyrrolidone copolymer has the structural Formula III:

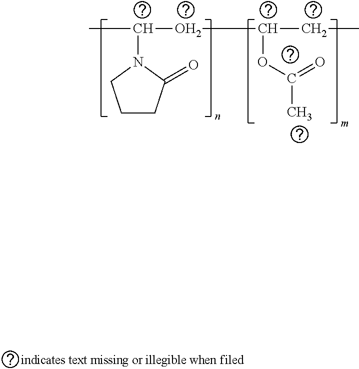

wherein n is about 1.2m.

The solid pharmaceutical formulation disclosed herein can further comprise an amphiphilic polymer. Amphiphilic polymers have both hydrophilic and lipophilic properties and are well-known in the art. Suitable examples of amphiphilic polymers include block and graft co-polymers such as, polyvinyl caprolactam-polyvinyl acetate-poly ethylene glycol graft copolymer (SOLUPLUS) and polymers referred to as Poloxamers (poly(ethylene glycol)-block-poly(propylene glycol)-block-poly(ethylene glycol), including Poloxamer 188, derivatives of polyacrylamides and cellulose and the like. See, e.g., Bezzaoucha et al., European Polymer J 2007 43(10)4440-4452 and Dong et al. Biomacromolecules 2016 17(2)454-465.

A preferred amphiphilic polymer is polyvinyl caprolactam-polyvinyl acetate-polyethylene glycol graft co-polymer. The amphiphilic polymer can have a structural formula as defined by Formula IV

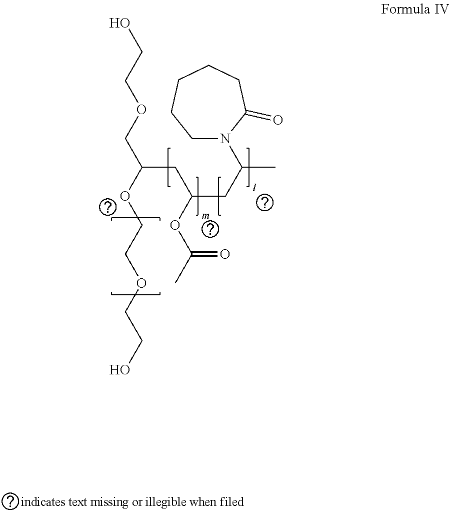

wherein PEG 6000/vinylcaprolactam/vinyl acetate is 13/57/30.

The amphiphilic polymer is more preferably Soluplus@ (polyvinyl caprolactam-polyvinyl acetate-polyethylene glycol graft copolymer, BASF).

Accordingly, in preferred embodiments, the solid pharmaceutical formulation includes a polymer matrix that comprises Kollidon V64 and SOLUPLUS.

The polymers used in the formulation of this disclosure can be of any desired molecular weight (weight average molecular weight), and typically have a weight average molecular weight of about 30,000 to about 150,000. All subranges of about 30,000 to about 150,000 are also included in this disclosure. For example, the polymer (e.g., neutral water-soluble polymer, amphiphilic polymer) can have a weight average molecular weight of about 45,000 to about 70,000, about 50,000 to about 80,000, about 40,000 to about 75,000, or about 30,000 to about 80,000, 90,000 to about 140,000, about 80,000 to about 110,000, about 90,000 to about 100,000, or about 100,000 to about 140,0000.

In particular examples, the polymer matrix includes a neutral water-soluble polymer that has a weight average molecular weight of about 30,000, about 35,000, about 40,000, about 45,000, about 50,000, about 55,000, about 60,000, about 65,000, about 70,000, about 75,000, about 80,000, about 85,000, about 90,000, about 95,000, about 100,000, about 115,000, about 120,000, about 125,000, about 130,000, about 135,000, about 140,000, about 145,000, about 150,000, and/or an amphiphilic polymer that has a weight average molecular weight of about 90,000 to about 140,000, about 80,000 to about 110,000, about 90,000 to about 100,000, or about 100,000 to about 140,0000.

While neutral water-soluble polymers are preferable for the formulations disclosed herein, it will be appreciated that the formulation can be formulated with other polymers, such as water soluble polymers, including nonionic water-soluble polymers and anionic water-soluble polymers.

b. Amorphous Neurosteroid Formulations

This disclosure relates to an solid pharmaceutical formulation comprising an amorphous neurosteroid, preferably ganaxolone, that is dispersed in a polymer matrix. The formulation is suitable for oral administration.

The neurosteroid, preferably ganaxolone, is substantially amorphous in the solid formulation. It is preferred that the sold pharmaceutical formulation contains substantially no crystalline Neurosteroid (ganaxolone) as determined by X-ray powder diffraction (XRPD). However, if desired, the sold pharmaceutical formulation can include some crystalline neurosteroid. In such embodiments, it is preferred that the neurosteroid (ganaxolone) in the solid pharmaceutical formulation is no more than about 30% crystalline, less than about 20% crystalline, less than about 15% crystalline, less than about 10% crystalline, less than about 5% crystalline, less than about 4% crystalline, less than about 3% crystalline, less than about 2% crystalline, or less than about 1% crystalline, as determined by XRPD. For example, the solid pharmaceutical formulation can include Ganaxolone that is not more than about 30% crystalline, less than about 29% crystalline, less than about 28% crystalline, less than about 27% crystalline, less than about 26% crystalline, less than about 25% crystalline, less than about 24% crystalline, less than about 23% crystalline, less than about 22% crystalline, less than about 21% crystalline, less than about 20% crystalline, less than about 19%, less than about 18% crystalline, less than about 17% crystalline, less than about 16% crystalline, less than about 15% crystalline, less than about 14% crystalline, less than about 13% crystalline, less than about 12% crystalline, less than about 11% crystalline, less than about 10% crystalline, less than about 9% crystalline, less than about 8% crystalline, less than about 7% crystalline, less than about 6% crystalline, less than about 5% crystalline, less than about 4% crystalline, less than about 3% crystalline, less than about 2% crystalline, or less than about 1% crystalline, as determined by XRPD.

The neurosteroid, e.g., ganaxolone can be about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amorphous, as determined by XRPD. Preferably, the neurosteroid, e.g., ganaxolone, is greater than about 70%, greater than about 80%, greater than about 90%, greater than about 95% amorphous, as determined by XPRD.

The neurosteroid, e.g., ganaxolone, can be less than about 30%, less than about 20%, less than about 15%, less than about 10%, less than about 5%, less than about 4%, less than about 3%, less than about 2%, or less than about 1% crystalline, as determined by XRPD. For example, the neurosteroid, e.g., ganaxolone, can be less than about 30%, less than about 29%, less than about 28%, less than about 27%, less than about 26%, less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, or less than about 1% crystalline, as determined by XRPD.

As another example, the neurosteroid, e.g., ganaxolone, can be no more than about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1%, crystalline as determined by XRPD.

The neurosteroid, e.g., ganaxolone can be no more than about 30% to about 1%, no more than about 30% to about 2%, no more than about 30% to about 3%, no more than about 30% to about 4%, no more than about 30% to about 5%, no more than about 30% to about 10%, no more than about 30% to about 15%, no more than about 30% to about 20%, no more than about 30% to about 25%, crystalline, as determined by XRPD. The neurosteroid, e.g., ganaxolone can be no more than about 20% to about 1%, no more than about 20% to about 2%, no more than about 20% to about 3%, no more than about 20% to about 4%, no more than about 20% to about 5%, no more than about 20% to about 10%, no more than about 20% to about 15% crystalline, as determined by XRPD. The neurosteroid, e.g., ganaxolone can be no more than about 15% to about 1%, no more than about 15% to about 2%, no more than about 15% to about 3%, no more than about 15% to about 4%, no more than about 15% to about 5%, no more than about 15% to about 10% crystalline, as determined by XRPD. The neurosteroid, e.g., ganaxolone can be no more than about 10% to about 1%, no more than about 10% to about 2%, no more than about 10% to about 3%, no more than about 10% to about 4%, no more than about 10% to about 5% crystalline, as determined by XRPD.

Ganaxolone can be about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% amorphous, as determined by XRPD. Preferably, ganaxolone, is greater than about 70%, greater than about 80%, greater than about 90%, greater than about 95% amorphous, as determined by XPRD.

Ganaxolone can be less than about 30%, less than about 20%, less than about 15%, less than about 10%, less than about 5%, less than about 4%, less than about 3%, less than about 2%, or less than about 1% crystalline, as determined by XRPD. For example, ganaxolone, can be less than about 30%, less than about 29%, less than about 28%, less than about 27%, less than about 26%, less than about 25%, less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, or less than about 1% crystalline, as determined by XRPD.

As another example, ganaxolone can be no more than about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1%, crystalline as determined by XRPD.

Ganaxolone can be no more than about 30% to about 1%, no more than about 30% to about 2%, no more than about 30% to about 3%, no more than about 30% to about 4%, no more than about 30% to about 5%, no more than about 30% to about 10%, no more than about 30% to about 15%, no more than about 30% to about 20%, no more than about 30% to about 25%, crystalline, as determined by XRPD. Ganaxolone can be no more than about 20% to about 1%, no more than about 20% to about 2%, no more than about 20% to about 3%, no more than about 20% to about 4%, no more than about 20% to about 5%, no more than about 20% to about 10%, no more than about 20% to about 15% crystalline, as determined by XRPD. Ganaxolone can be no more than about 15% to about 1%, no more than about 15% to about 2%, no more than about 15% to about 3%, no more than about 15% to about 4%, no more than about 15% to about 5%, no more than about 15% to about 10% crystalline, as determined by XRPD. Ganaxolone can be no more than about 10% to about 1%, no more than about 10% to about 2%, no more than about 10% to about 3%, no more than about 10% to about 4%, no more than about 10% to about 5% crystalline, as determined by XRPD.

The state of the solid pharmaceutical formulation can be further confirmed by XRPD.

The neurosteroid, e.g., ganaxolone can be present in an amount of about 10% to about 60% by weight with respect to the solid pharmaceutical formulation. The neurosteroid, e.g., ganaxolone can be present in an amount of about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40% by weight with respect to the solid pharmaceutical formulation. Typically, the neurosteroid (e.g., ganaxolone) is present in an amount between about 20% and about 30% by weight with respect to the solid pharmaceutical formulation.

Ganaxolone can be present in an amount of about 10% to about 60% by weight with respect to the solid pharmaceutical formulation. Ganaxolone can be present in an amount of about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40% by weight with respect to the solid pharmaceutical formulation. Typically, ganaxolone is present in an amount between about 20% and about 30% by weight with respect to the solid pharmaceutical formulation.

The polymer (or polymer mixture) to neurosteroid (e.g., ganaxolone) ratio in the solid pharmaceutical formulation can be about 90:1 to about 2:1 by weight. Preferably, the polymer (or polymer mixture) to neurosteroid (e.g., ganaxolone) ratio in the solid pharmaceutical formulation can be about 9:1 to about 7:3 weight, such as about 4:1 by weight. Accordingly, in embodiments, the solid pharmaceutical formulation can contain about 90% to about 70% polymer (or polymer mixture) and about 10% to about 30% neurosteroid (ganaxolone) by weight, for example, about 80% polymer (or polymer mixture) and about 20% ganaxolone by weight.

The solid pharmaceutical formulation can contain about 50% to about 90% polymer or polymer mixture (that make up the polymer matrix) by weight. For example, the solid pharmaceutical formulation can include a neutral water-soluble polymer in an amount from about 50% to about 90% by weight with respect to the solid pharmaceutical formulation. The neutral water-soluble polymer can be present in an amount of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90% by weight with respect to the solid pharmaceutical formulation. Typically, the neutral water-soluble polymer is present in an amount between about 50% to about 80% by weight with respect to the solid pharmaceutical formulation. For example, the neutral water-soluble polymer can be present in an amount of about 55% by weight with respect to the solid pharmaceutical formulation. For example, the neutral water-soluble polymer can be present in an amount of about 70% by weight with respect to the solid pharmaceutical formulation. For example, the neutral water-soluble polymer can be present in an amount of about 80% by weight.

The amphiphilic polymer can be in an amount of about 1% to about 30% by weight with respect to the solid pharmaceutical formulation. The amphiphilic polymer can be present in an amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight with respect to the solid pharmaceutical formulation. Typically, the amphiphilic polymer is present in an amount of about 20% to about 25% by weight with respect to the solid pharmaceutical formulation. If desired, the amphiphilic polymer can be present in the solid pharmaceutical formulations disclosed herein. The amphiphilic polymer may not be present in the solid pharmaceutical formulations disclosed herein.

The neutral water-soluble polymer can be in a w/w ratio to neurosteroid of about 50:10, about 50:15, about 50:20, about 50:25, about 50:30, about 50:35, about 50:40, about 55:10, about 55:15, about 55:20, about 55:25, about 55:30, about 55:35, about 55:40, about 60:10, about 60:15, about 60:20, about 60:25, about 60:30, about 60:35, about 60:40, about 65:10, about 65:15, about 65:20, about 65:25, about 65:30, about 65:35, about 65:40, about 70:10, about 70:15, about 70:20, about 70:25, about 70:30, about 70:35, about 70:40, about 75:10, about 75:15, about 75:20, about 75:25, about 75:30, about 75:35, about 75:40, about 80:10, about 80:15, about 80:20, about 80:25, about 80:30, about 80:35, about 80:40, about 90:10, about 90:15, about 90:20, about 90:25, about 90:30, or greater. All ranges including any of the two foregoing w/w ratio of the neutral water-soluble polymer to neurosteroid are also included in the disclosure. For example, the disclosure includes a w/w ratio of the neutral water-soluble polymer to neurosteroid of about 50:10 to about 90:40, about 60:10 to about 80:40, about 70:10 to about 80:20. Preferably, the w/w ratio of the neutral water-soluble polymer to ganaxolone is about 70:30. Preferably, the w/w ratio of the neutral water-soluble polymer to ganaxolone is about 80:20.

The neutral water-soluble polymer can be in a w/w ratio to ganaxolone of about 50:10, about 50:15, about 50:20, about 50:25, about 50:30, about 50:35, about 50:40, about 55:10, about 55:15, about 55:20, about 55:25, about 55:30, about 55:35, about 55:40, about 60:10, about 60:15, about 60:20, about 60:25, about 60:30, about 60:35, about 60:40, about 65:10, about 65:15, about 65:20, about 65:25, about 65:30, about 65:35, about 65:40, about 70:10, about 70:15, about 70:20, about 70:25, about 70:30, about 70:35, about 70:40, about 75:10, about 75:15, about 75:20, about 75:25, about 75:30, about 75:35, about 75:40, about 80:10, about 80:15, about 80:20, about 80:25, about 80:30, about 80:35, about 80:40, about 90:10, about 90:15, about 90:20, about 90:25, about 90:30, or greater. All ranges including any of the two foregoing w/w ratio of neutral water-soluble polymer to ganaxolone are also included in the disclosure. For example, the disclosure includes a w/w ratio of the neutral water-soluble polymer to ganaxolone of about 50:10 to about 90:40, about 60:10 to about 80:40, about 70:10 to about 80:20. Preferably, the w/w ratio of the neutral water-soluble polymer to ganaxolone is about 70:30. Preferably, the w/w ratio of the neutral water-soluble polymer to ganaxolone is about 80:20.

The neutral water-soluble polymer can be in a w/w ratio to amphiphilic polymer to neurosteroid can be about 50:1:10, about 50:5:10, about 50:10:10, about 50:15:10, about 50:20:10, about 50:25:10, about 50:30:10, about 50:35:10, about 50:40:10, 50:1:20, about 50:5:20, about 50:10:20, about 50:15:20, about 50:20:20, about 50:25:20, about 50:30:20, about 50:35:20, about 50:40:20, about 50:45:20, about 50:50:20, 50:1:30, about 50:5:30, about 50:10:30, about 50:15:30, 50:1:40, about 50:5:40, about 50:10:40, 60:1:10, about 60:5:10, about 60:10:10, about 60:15:10, about 60:20:10, about 60:25:10, about 60:30:10, 60:1:20, about 60:5:20, about 60:10:20, about 60:15:20, about 60:20:20, 60:1:30, about 60:5:30, about 60:10:30, 70:1:10, about 70:5:10, about 70:10:10, about 70:15:10, about 70:20:10, about 70:1:20, about 70:5:20, about 70:10:20, 80:1:19, about 80:5:15, about 80:10:10 or greater. All ranges including any of the two foregoing w/w ratio of neutral water-soluble polymer to amphiphilic polymer to neurosteroid are also included in the disclosure. For example, the disclosure includes a w/w ratio of the neutral water-soluble polymer to amphiphilic polymer to neurosteroid of about 50:10:1 to about 50:40:10, about 60:10:10 to about 70:25:5, about 50:20:30 to about 60:20:20. Preferably, the w/w ratio of the neutral water-soluble polymer to amphiphilic polymer to neurosteroid is about 56:24:20. Preferably, the w/w ratio of the neutral water-soluble polymer to amphiphilic polymer to neurosteroid is about 50:20:30.

The neutral water-soluble polymer can be in a w/w ratio to amphiphilic polymer to ganaxolone can be about 50:1:10, about 50:5:10, about 50:10:10, about 50:15:10, about 50:20:10, about 50:25:10, about 50:30:10, about 50:35:10, about 50:40:10, 50:1:20, about 50:5:20, about 50:10:20, about 50:15:20, about 50:20:20, about 50:25:20, about 50:30:20, about 50:35:20, about 50:40:20, about 50:45:20, about 50:50:20, 50:1:30, about 50:5:30, about 50:10:30, about 50:15:30, 50:1:40, about 50:5:40, about 50:10:40, 60:1:10, about 60:5:10, about 60:10:10, about 60:15:10, about 60:20:10, about 60:25:10, about 60:30:10, 60:1:20, about 60:5:20, about 60:10:20, about 60:15:20, about 60:20:20, 60:1:30, about 60:5:30, about 60:10:30, 70:1:10, about 70:5:10, about 70:10:10, about 70:15:10, about 70:20:10, about 70:1:20, about 70:5:20, about 70:10:20, 80:1:19, about 80:5:15, about 80:10:10 or greater. All ranges including any of the two foregoing w/w ratio of neutral water-soluble polymer to amphiphilic polymer to ganaxolone are also included in the disclosure. For example, the disclosure includes a w/w ratio of the neutral water-soluble polymer to amphiphilic polymer to ganaxolone of about 50:10:1 to about 50:40:10, about 60:10:10 to about 70:25:5, about 50:20:30 to about 60:20:20. Preferably, the w/w ratio of the neutral water-soluble polymer to amphiphilic polymer to ganaxolone is about 56:24:20. Preferably, the w/w ratio of the neutral water-soluble polymer to amphiphilic polymer to ganaxolone is about 50:20:30.

An exemplary formulation according to this disclosure can comprise ganaxolone in an amount of about 20% by weight with respect to the solid pharmaceutical formulation, the neutral water-soluble polymer in an amount of about 56% by weight with respect to the solid pharmaceutical formulation, and an amphiphilic polymer amphiphilic polymer in an amount of about 24% by weight with respect to the solid pharmaceutical formulation. As another example, the formulation can comprise ganaxolone in an amount of about 20% by weight with respect to the solid pharmaceutical formulation and a neutral water-soluble polymer in an amount of about 80% by weight with respect to the solid pharmaceutical formulation. As another example, the formulation can comprise ganaxolone in an amount of about 30% by weight with respect to the solid pharmaceutical formulation and a neutral water-soluble polymer in an amount of about 70% by weight with respect to the solid pharmaceutical formulation. As another example, the formulation can comprise ganaxolone in an amount of about 30% by weight with respect to the solid pharmaceutical formulation, a neutral water-soluble polymer in an amount of about 50% by weight with respect to the solid pharmaceutical formulation, and an amphiphilic polymer amphiphilic polymer in an amount of about 20% by weight with respect to the solid pharmaceutical formulation.

In embodiments, the solid pharmaceutical formulation comprises ganaxolone that is dispersed in a polymer matrix that contains KOLLIDON VA64, SOLUPLUS or a combination of KOLLIDON AV64 and SOLUPLUS. In such embodiments, the ganaxolone is substantially amorphous. In a particular examples of such embodiments the solid pharmaceutical formulation comprises Kollidon VA64, SOLUPLUS and ganaxolone, and the ratio Kollidon VA64:SOLUPLUS:ganaxolone is about 56:24:20 by weight percent. In another particular examples of such embodiments the solid pharmaceutical formulation comprises SOLUPLUS and ganaxolone, and the ratio SOLUPLUS:ganaxolone is about 80:20 by weight percent.

The solid pharmaceutical formulation can be in the form of a powder. The powder can comprise particles that have a volume weighted median diameter (Dv50) of about 50 nm to about 500 nm (Dv50).

The particle size can be between about 50 nm to about 2500 nm, between about 50 nm and 1000 n, between about 50 nm and 800 nm, between about 50 nm and 600 nm, between about 50 nm and 500 nm, between about 50 nm and 400 nm, between about 50 nm and 300 nm, between about 50 nm and 200 nm, or between about 100 nm and 300 nm (Dv50). In one embodiment, particle is about 50 to about 200 nm (Dv50). In one embodiment, the particle is about 100 nm (Dv50).

The solid pharmaceutical formulations disclosed herein are suitable for oral administration. The formulations disclosed herein are formulated as a solid dosage form. Exemplary solid dosage forms include oral solid dosage forms, aerosols, controlled release formulations, fast melt formulations, effervescent formulations, self-emulsifying dispersions, solid solutions, liposomal dispersions, lyophilized formulations, tablets, capsules, pills, powders, delayed release formulations, immediate release formulations, modified release formulations, extended release formulations, pulsatile release formulations, multiparticulate formulations, and mixed immediate release and controlled release formulations. The solid pharmaceutical formulation disclosed herein is preferably in the form of a powder.

The solid pharmaceutical formulation in the form of a powder can be added to any suitable food substrate. Exemplary food substrates include, but are not limited to, fruit puree (e.g., applesauce, ai, aceola, apple, apricots, avocado, banana, blackberry, blueberries, cantaloupe, camu berry, cherimoya, cherries, clementine, coconut, cranberry, cucumber, currents, dates, durian, fig, goji berries, gooseberry, grapefruit, grapes, jackfruit, jujube, kiwi, kumquat, lemon, lime, longan, loquat, lucuma, lychee, mamey sapote, mango, mangosteen, melon, mulberry, nectarine, orange, papaya, passion fruit, peach, pear, persimmon, pineapple, plum, pomegranate, pomelo, prickly pear, prunes, raspberries, sapote, strawberries, tangerine, tomato, watermelon, and combinations thereof), yoghurt, ice-cream, fruit, cheese, or gelato.

The powder can be sprinkled or mixed into suitable food substrates.

In embodiments, the solid pharmaceutical formulation disclosed herein can in the form of a tablet. The solid pharmaceutical formulation can be in the form of a capsule. The solid pharmaceutical formulation can be in the form of a single capsule dosage form or multiple capsule dosage forms (e.g., two, three, or four capsules).

The solid pharmaceutical formulations disclosed herein can further comprise one or more pharmaceutically acceptable excipients such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. For example, the solid pharmaceutical formulation can include surfactants and/or stabilizers, such as sodium lauryl sulfate (SLS), TPGS (D-alpha-tocopherol polyethylene glycol 1000 succinate), PEG1500 (polyethylene glycol 1500), Poloxamer 188 (poly(ethylene glycol)-block-poly(propylene glycol)-block-poly(ethylene glycol), valproic acid and the like. In still other aspects, using standard coating procedures, such as those described in Remington's Pharmaceutical Sciences, 20th Edition (2000), a film coating is provided around the ganaxolone formulation.

Tablets, troches, pills and capsules may also contain one or more of the following: a binder such as gum tragacanth, acacia, corn starch or gelatin; an excipient, such as dicalcium phosphate; a disintegrating agent such as corn starch, potato starch, alginic acid and the like; a lubricant such as magnesium stearate; a sweetening agent such as sucrose, lactose or saccharin; or a flavoring agent such as peppermint, oil of wintergreen or cherry flavoring. When the unit dosage form is a capsule, it may contain, in addition to materials of the above type, a liquid carrier. Various other materials may be present as coating, for instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. It may be desirable for material in a dosage form or pharmaceutical composition to be pharmaceutically pure and substantially non-toxic in the amounts employed. Some oral dosage forms may have enteric coatings or film coatings. In some embodiments, a dosage form may comprise a tablet or a capsule having an enteric coating. A dosage form may comprise a tablet or a capsule having a film coating.

Suitable carriers for use in the solid dosage forms described herein include, but are not limited to, acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropyhnethylcellulose acetate stearate, sucrose, microcrystalline cellulose, lactose, mannitol and the like. [00184] Suitable filling agents for use in the solid dosage forms described herein include, but are not limited to, lactose, calcium carbonate, calcium phosphate, dibasic calcium phosphate, calcium sulfate, microcrystalline cellulose (e.g., Avicel®, Avicel® PH101, Avicel® PH102, Avicel® PH105, etc.), cellulose powder, dextrose, dextrates, dextran, starches, pregelatinized starch, hydroxypropyhnethylcellulose (HPMC), hydroxypropylmethylcellulose phthalate, hydroxypropyhnethylcellulose acetate stearate (HPMCAS), sucrose, xylitol, lactitol, mannitol, sorbitol, sodium chloride, polyethylene glycol, and the like.

Binders impart cohesiveness to solid oral dosage form formulations: for powder filled capsule formulation, they aid in plug formation that can be filled into soft or hard shell capsules and in tablet formulation, binders ensure that the tablet remains intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Agoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crosspovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-IO, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations is a function of whether direct compression, wet granulation, roller compaction, or usage of other excipients such as fillers which itself can act as moderate binder are used.

Suitable lubricants or glidants for use in the solid dosage forms described herein include, but are not limited to, stearic acid, calcium hydroxide, tale, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable suspending agents for use in the solid dosage forms described here include, but are not limited to, polyvinylpyrrolidone, e.g., polyvinylpyrrolidone K 1 2, polyvinylpyrrolidone K 1 7, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 18000, vinyl pyrrolidone/vinyl acetate copolymer (S630), sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, polyethoxylated sorbitan monolaurate, polyethoxylated sorbitan monolaurate, povidone and the like.

The solid pharmaceutical formulations disclosed herein can optionally include a flavoring agent (e.g., a natural or artificial sweetening agent). Examples of natural and artificial sweetening agents suitable for the formulations described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

The formulation can comprise a sweetening agent or flavoring agent in a concentration ranging from about 0.0001% to about 10.0% the weight of the aqueous dispersion. The formulation can comprise a sweetening agent or flavoring agent in a concentration ranging from about 0.0005% to about 5.0% wt % of the aqueous dispersion. The formulation can comprise a sweetening agent or flavoring agent in a concentration ranging from about 0.0001% to 0.1 wt %, from about 0.001% to about 0.01 weight %, or from 0.0005% to 0.004% of the solid pharmaceutic formulation.

The solid pharmaceutical formulations disclosed herein are suitable for oral administration. While the formulations disclosed herein are formulated in the form of a solid dosage form, it will be appreciated that the formulations disclosed herein can be formulated for other oral dosage forms, for example, aqueous oral suspensions or liquid dosage forms.

The solid pharmaceutical formulations disclosed herein may be dissolved or reconstituted in water or other suitable liquid to form an oral liquid formulation. An oral liquid formulation (e.g., the solid pharmaceutical formulation dissolved in water), can comprise a buffering agent. For example, an acetate, citrate, tartrate, phosphate, or triethanolamine (TRIS) buffer, or an acid or base buffer to adjust pH to desired levels. An oral liquid formulation can have a pH of about 2.5 to 11, 3.5 to 9.0, or 5.0 to 8.0, or 6.0 to 8.0, or 6.0 to 7.0, or 6.2 to 7.10. The formulation disclosed herein can have a pH of about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, or about 11. The formulation disclosed herein preferably has a pH of about 6.0 to 7.0. For example, a pH of 6.0, 6.1, 6.2, 6.3, 6.4, 6.5, 6.6, 6.7, 6.8, 6.9, 7.0.

Examples of acid buffers useful in liquid dosage formulations include oxalic acid, maleic acid, fumaric acid, lactic acid, malic acid, tartaric acid, citric acid, benzoic acid, acetic acid, metbanesulfonic acid, histidine, succinic acid, toluenesulfonic acid, benzenesulfonic acid, ethanesulfonic acid and the like. Acid salts of the above acids may be employed as well. Examples of base buffers useful in liquid dosage formulations include carbonic acid and bicarbonate systems such as sodium carbonate and sodium bicarbonate, and phosphate buffer systems, such as sodium monohydrogen phosphate and sodium dihydrogen phosphate. The buffer can be a phosphate buffer. In embodiments, the buffer can be phosphate buffered saline. The buffer can be a mixture of monobasic and dibasic phosphate buffers. For example, potassium phosphate mono or dibasic phosphate buffers. The concentration of each component of a phosphate buffer system can be from about 1 mM to about 20 mM, about 10 mM to about 200 mM, or from about 20 mM to about 150 mM, or from about 50 mM to about 100 mM.

A liquid formulation comprising the solid pharmaceutical formulations disclosed herein can contain electrolytes, such as sodium or potassium. The disclosure includes embodiments in which the formulation is from about 0.05% to about 1.5% sodium chloride (saline).

Liquid dosage formulations can optionally further comprise one or more pharmaceutically acceptable additives, such as a compatible carrier, binder, complexing agent, ionic dispersion modulator, filing agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combinations.

Liquid formulations can be prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, disintegrators, fillers, matrix-forming compositions and coating compositions. “Carrier” also includes all components of the coating composition which may include plasticizers, pigments, colorants, stabilizing agents, and glidants.

Pharmaceutically acceptable excipients include, but not limited to, surfactants, dispersants, emulsifiers, pH modifying agents, and combination thereof. Suitable surfactants may be anionic, cationic, amphoteric or nonionic surface active agents. Suitable anionic surfactants include, but are not limited to, those containing or comprising carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl- -alanine, sodium N-lauryl--iminodipropionate, myristoamphoacetate, lauryl betaine and lauryl sulfobetaine.

C. Methods of Preparing the Solid Pharmaceutical Formulations

The solid pharmaceutical formulations can be prepared using any suitable method. Many suitable methods for preparing the solid pharmaceutical formulations disclosed herein are conventional in the art, and include hot melt extrusion, single and double emulsion solvent evaporation, spray drying, spray freeze drying, milling (e.g., jet milling), blending, solvent extraction, solvent evaporation, phase separation, simple and complex coacervation, supercritical fluid-based processes, and other suitable methods, including combinations thereof.

The solid pharmaceutical formulations disclosed herein can be prepared using a hot melt extrusion technique.

The pharmaceutical formulation disclosed herein may be loaded into a hot melt extruder, heated to a temperature above the glass transition temperature, and extruded using a light compressive force to push the material through the nozzle and a light tensile force to pull the material out of the extruder. The extrudate may be milled. The extrudate can be cut to the desired length for appropriate drug dosing.

A milling process can be used to reduce the size of the extrudate to form sized particles, e.g., beads, in the micrometer (microbeads) to nanometer size range (nanobeads). The milling process may be performed using a mill or other suitable apparatus. Dry and wet milling processes such as jet milling, cryo-milling, ball milling, media milling, sonication, and homogenization are known and can be used in methods described herein. Generally, in a wet milling process, a suspension of the material to be used as the core is agitated with or without excipients to reduce particle size. Dry milling is a process wherein the material to be used as the article core is mixed with milling media with or without excipients to reduce particle size. In a cyro-milling process, a suspension of the material to be used as the core is mixed with milling media with or without excipients under cooled temperatures.

To prepare the solid pharmaceutical formulations disclosed herein, generally, the components of the solid pharmaceutical formulations disclosed herein, for example, the neurosteroid, e.g., ganaxolone, the neutral water-soluble polymer and/or the optional amphiphilic polymer are loaded into a hot melt extruder. The hot melt process involves applying agitation to blend the components, heated to a temperature above the glass transition temperature to achieve a melted product and extruded using a light compressive force to push the material through the nozzle and a light tensile force to pull the material out of the extruder. The extrudate is generally milled to form a powder.

The solid pharmaceutical formulations can be prepared using a spray drying technique. Suitable spray drying techniques are described, for example, by K. Masters in “Spray Drying Handbook”, John Wiley & Sons, New York (1984). Generally, during spray drying, heat from a hot gas such as heated air or nitrogen is used to evaporate a solvent from droplets formed by atomizing a continuous liquid feed. If desired, the spray drying or other instruments, e.g., jet milling instrument, used to prepare the dry particles can include an inline geometric particle sizer that determines a geometric diameter of the respirable dry particles as they are being produced, and/or an inline aerodynamic particle sizer that determines the aerodynamic diameter of the respirable dry particles as they are being produced.

For spray drying, solutions, emulsions or suspensions that contain the components of the solid pharmaceutical compositions disclosed herein can be distributed to a drying vessel via an atomization device. For example, a nozzle or a rotary atomizer may be used to distribute the solution or suspension to the drying vessel. For example, a rotary atomizer having a 4- or 24-vaned wheel may be used. Examples of suitable spray dryers that can be outfitted with either a rotary atomizer or a nozzle, include a Mobile Minor Spray Dryer or the Model PSD-1, both manufactured by GEA Group (Niro, Denmark), B0chi B-290 Mini Spray Dryer (BUCHI Labortechnik AG, Flawil, Switzerland), ProCepT Formatrix R&D spray dryer (ProCepT nv, Zelzate, Belgium), among several other spray dryer options. Actual spray drying conditions will vary depending, in part, on the composition of the spray drying solution or suspension and material flow rates. The person of ordinary skill will be able to determine appropriate conditions based on the compositions of the solution, emulsion or suspension to be spray dried, the desired particle properties and other factors. In general, the inlet temperature to the spray dryer is about 90° C. to about 300° C., and preferably is about 220° C. to about 285° C. The spray dryer outlet temperature will vary depending upon such factors as the feed temperature and the properties of the materials being dried. If desired, the solid pharmaceutical formulation that is produced can be fractionated by volumetric size, for example, using a sieve, or fractioned by aerodynamic size, for example, using a cyclone, and/or further separated according to density using techniques known to those of skill in the art.

The solid pharmaceutical formulations can be prepared using supercritical fluid technology.

D. Pharmacokinetics

The solid pharmaceutical formulations described herein can have enhanced pharmacokinetic (PK) and pharmacodynamic (PR) profiled and/minimized side effects as compared to the oral ganaxolone nanosuspension formulation described in the art (see, International Application No. PCT/US2006/045626). Specifically, the solid pharmaceutical formulations described herein can provide increased therapeutic benefit resulting from the enhanced PK/PD properties. The amorphous neurosteroid (e.g., ganaxolone) in a polymer matrix appears to substantially increase the oral bioavailability of ganaxolone after oral administration to the subject, such as a human. Without being bound by theory, it is believed that the increased bioavailability increases exposure of the neurosteroid (e.g., ganaxolone) and/or improves maintenance of the neurosteroid (e.g., ganaxolone) steady state. The amorphous neurosteroid dispersed in a polymer matrix may also substantially increase the solubility and rate of absorption of ganaxolone after oral administration in comparison to other Ganaxolone formulations.

The solid pharmaceutical formulations disclosed herein can reduce the risk of ganaxolone side effects, including ataxia, sedation, somnolence, and anesthesia due to the improved PK/PD properties, such as improved bioavailability.

The solid pharmaceutical formulations disclosed herein can improve the oral bioavailability of ganaxolone by at least about 5%, about 10%, about 15%, about 20%, about 30%, about 40%, about 50%, about 60%, about 70, about 80%, about 80%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 325%, about 350%, about 375%, about 400%, about 425%, about 450%, about 475%, about 500%, about 525%, about 550%, about 575%, about 600%, about 625%, about 650%, about 675%, about 700%, about 725%, about 750%, about 775%, about 800%, about 825%, about 850%, about 875%, about 900%, about 925%, about 950%, about 975%, about 1000% or greater, or any amount in a range bounded by, or between, these values for example, as compared to ganaxolone nanosuspension formulation.

The enhanced bioavailability achieved by the solid pharmaceutical formulations disclosed herein can be measured by the area under the curve (AUC) level. In embodiments, the solid pharmaceutical formulations disclosed herein may increase the Cmax and/or the AUC, e.g., in comparison to a ganaxolone suspension.

The solid pharmaceutical formulations described herein may result in a maximum ganaxolone plasma concentration (Cmax) from about 100 ng/ml to about 1200 ng/ml. For example, the ganaxolone formulation may achieve a Cmax of about 100 ng/ml, about 110 ng/ml, about 120 ng/ml, about 130 ng/ml, about 140 ng/ml, about 150 ng/ml, about 160 ng/ml, about 170 ng/ml, about 180 ng/ml, about 190 ng/ml, about 200 ng/ml, about 210 ng/ml, about 220 ng/ml, about 230 ng/ml, about 240 ng/ml, about 250 ng/ml, about 260 ng/ml, about 270 ng/ml, about 280 ng/ml, about 290 ng/ml, about 300 ng/ml, about 310 ng/ml, about 320 ng/ml, about 330 ng/ml, about 340 ng/ml, about 350 ng/ml, about 360 ng/ml, about 370 ng/ml, about 380 ng/ml, about 390 ng/ml, about 400 ng/ml, about 410 ng/ml, about 420 ng/ml, about 430 ng/ml, about 440 ng/ml, about 450 ng/ml, about 460 ng/ml, about 470 ng/ml, about 480 ng/ml, about 490 ng/ml, about 500 ng/ml, about 510 ng/ml, about 520 ng/ml, about 530 ng/ml, about 540 ng/ml, about 550 ng/ml, about 560 ng/ml, 570 ng/ml, about 580 ng/ml, about 590 ng/ml, about 600 ng/ml, about 610 ng/ml, about 620 ng/ml, about 630 ng/ml, about 640 ng/ml, about 650 ng/ml, about 660 ng/ml, about 670 ng/ml, about 680 ng/ml, about 690 ng/ml, about 700 ng/ml, about 710 ng/ml, about 720 ng/ml, about 730 ng/ml, about 740 ng/ml, about 750 ng/ml, about 760 ng/ml, about 770 ng/ml, about 780 ng/ml, about 790 ng/ml, 800 ng/ml, about 810 ng/ml, about 820 ng/ml, about 830 ng/ml, about 840 ng/ml, about 850 ng/ml, about 900 ng/ml, about 910 ng/ml, about 920 ng/ml, about 930 ng/ml, 940 ng/ml, about 950 ng/ml, about 960 ng/ml, about 970 ng/ml, about 980 ng/ml, about 990 ng/ml, about 1000 ng/ml, about 1100 ng/ml, or about 1200 ng/ml.

The solid pharmaceutical formulations described herein can increase ganaxolone Cmax by at least about 1 fold, about 2 fold, about 3 fold, about 4 fold, about 5 fold, about 6 fold, about 7 fold, about 8 fold, about 9 fold, about 10 fold, about 11 fold, about 12 fold, about 13 fold, about 15 fold, about 16 fold, about 17 fold, about 18 fold, about 19 fold, about 20 fold, or greater for the same ganaxolone dose administered as an oral ganaxolone nanosuspension formulation as described International Application No. PCT/US2006/045626.

The solid pharmaceutical formulations disclosed herein can increase the AUC of ganaxolone. For example, oral administration of the solid pharmaceutical formulations disclosed comprising amorphous ganaxolone dispersed in a polymer matrix can result in an AUC of ganaxolone of about 100 ng/ml/hr to about 1,200 ng/ml/hr, about 200 ng/ml/hr to about 1,200 ng/ml/hr, about 300 ng/ml/hr to about 1,200 ng/ml/hr, about 400 ng/ml/hr to about 1,200 ng/ml/hr, about 500 ng/ml/hr to about 1,200 ng/ml/hr, about 600 ng/ml/hr to about 1,200 ng/ml/hr, about 700 ng/ml/hr to about 1,200 ng/ml/hr, about 800 ng/ml/hr to about 1,200 ng/ml/hr, about 900 ng/ml/hr to about 1,200 ng/ml/hr, or any AUC in a range bounded by, or between, these values. For example, the AUC can be the AUC calculated to the last measured concentration (AUC0.t), over a period of 24 hours (AUC0-24).

In embodiments, the solid pharmaceutical formulations disclosed herein can be administered at a dose that results in an average ganaxolone plasma concentration (Cave) of about 100 ng/ml to about 1,200 ng/ml, about 200 ng/ml to about 1,200 ng/ml, about 300 ng/ml to about 1,200 ng/ml, about 400 ng/ml to about 1,200 ng/ml, about 500 ng/ml to about 1,200 ng/ml, about 600 ng/ml to about 1,200 ng/ml, about 700 ng/ml to about 1,200 ng/ml, about 800 ng/ml to about 1,200 ng/ml, about 900 ng/ml to about 1,200 ng/ml, or any Cave in a range bounded by, or between these values.

The solid pharmaceutical formulations disclosed herein can be administered at a dose that results in plasma ganaxolone concentration of about 100 ng/ml to about 1,200 ng/ml, about 200 ng/ml to about 1,200 ng/ml, about 300 ng/ml to about 1,200 ng/ml, about 400 ng/ml to about 1,200 ng/ml, about 500 ng/ml to about 1,200 ng/ml, about 600 ng/ml to about 1,200 ng/ml, about 700 ng/ml to about 1,200 ng/ml, about 800 ng/ml to about 1,200 ng/ml, about 900 ng/ml to about 1,200 ng/ml, or any ganaxolone plasma concentration in a range bounded by, or between any of these values.

E. Method of Treating

The disclosure also relates methods for treating a disorder. The methods disclosed herein comprise orally administering to a subject in need thereof a therapeutically effective amount of the solid pharmaceutical formulations disclosed herein comprising an amorphous neurosteroid, preferably ganaxolone dispersed in a polymer matrix.

The methods can be suitable for treating any type of seizure disorder, epilepsy disorders, genetic epilepsy disorders, epilepsy related disorders, central nervous system disorders, neurological disorders, and neurodegenerative disorders.

Types of epilepsy disorders and/or epilepsy related disorders the methods disclosed herein can be suitable for treating include, but are not limited to, pediatric epilepsy, SCN8 A epilepsy, catamenial epilepsy, Angelman syndrome, benign epilepsy with centro-temporal spikes (BECTS), CDKL5 deficiency disorder, autosomal dominant nocturnal frontal lobe epilepsy (ADFE), absence epilepsy, childhood absence epilepsy, Doose syndrome, Dravet syndrome, early myoclonic epilepsy (EME), epilepsy with generalized tonic-clonic seizures, epilepsy with myoclonic-absenses, infantile spasms (West syndrome), Landau-Kleffner syndrome, Lennox-Gastaut syndrome, epilepsy with myoclonic absences, frontal lobe epilepsy, juvenile myoclonic epilepsy, Lyfora progressive myoclonus epilepsy, Ohtahara syndrome, Panayiotopoulos syndrome, Rasmussen's syndrome, progressive myoclonic epilepsies, Ring chromosome 20 syndrome, temporal lobe epilepsy, epilepsy of infancy with migrating focal seizures, epilepsy in Fragile X syndrome, Sturge-Weber Syndrome, PCDH19-related epilepsy, Tuberous Scelrosis Complex, Dupl5q Syndrome, Jeavons Syndrome, Febrile Illness-Related Epilepsy Syndrome (FIRES), Autoimmune Encephalitis/Encephalopathy due to Anti-LGI1, Anti-NMDAR, Anti-Gaba-B receptor, or Anti-GAD65 antibodies, FOXG1-related epilepsy, Syngap 1-related epilepsy, Landau-Kleffner syndrome, Aircardi Syndrome, Otahara Syndrome, Sleep-related Hypermotor Epilepsy, Epileptic Encephalopathy with Continuous Spike and Wave During Sleep (CSWS), Myoclonic Epilepsy of Infancy, Rasmussen encephalitis/syndrome, Developmental and Epileptic Encephalopathies (unclassified), Focal Epilepsy, Frontal Lobe Epilepsy, Temporal Lobe Epilepsy, Juvenile Absence Epilepsy, Posttraumatic Epilepsy, Epilepsy due to Cortical Dysplasia, and Catamenial Epilepsy.

Additional medical conditions manifesting with seizures that can be suitable for the methods disclosed herein include, but are not limited to preeclampsia/eclampsia, drug related seizures (including prescriptions drugs uses as prescribed (e.g., antihistamines, antibiotics, antidepressants, antipsychotics), illicit substance overdoses (PCP, cocaine, amphetamines), drug withdrawal (THC, opioids, alcohol, barbiturates) or AED non-compliance.

Epilepsy disorders and/or epilepsy related disorders that are particularly suitable for the methods disclosed herein include CDKL5 Deficiency Disorder, Tuberous Scelrosis Complex, and PCDH19-related epilepsy.

The methods disclosed herein are suitable for treating seizures, such as status epileptics. The methods disclosed herein are suitable for any form of SE. For example, early status epilepticus, established status epilepticus, refractory status epilepticus, super-refractory status epilepticus, non-convulsive status epilepticus (e.g., focal non-convulsive status epilepticus, complex partial non-convulsive status epilepticus, simple partial non-convulsive status epilepticus, subtle non-convulsive status epilepticus), generalized convulsive status epilepticus, complex partial status epilepticus; generalized periodic epileptiform discharges, and periodic lateralized epileptiform discharges.

Other forms of seizures the methods disclosed herein can be suitable for include epileptic seizures, acute repetitive seizures, cluster seizures, continuous seizures, unremitting seizures, prolonged seizures, recurrent seizures, refractory seizures, myoclonic seizures, tonic seizures, tonic-clonic seizures, simple partial seizures, complex partial seizures, secondarily generalized seizures, atypical absence seizures, absence seizures; atonic seizures, benign Rolandic seizures, febrile seizures, emotional seizures, focal seizures, gelastic seizures, generalized onset seizures, infantile spasms, Jacksonian seizures, massive bilateral myoclonus seizures, multifocal seizures, neonatal onset seizures, nocturnal seizures, occipital lobe seizures, post traumatic seizures, subtle seizures, Sylvan seizures, visual reflex seizures, seizures from traumatic brain injury, seizures resulting from exposure to chemical weapons (e.g., organophosphate nerve gas or withdrawal seizures).

The formulations disclosed herein can also be administered as a prophylactic for treating central nervous system disorders and/or neurological disorders. Exemplary central nervous system disorders and/or neurological disorders that may be suitable for treating using the formulations disclosed herein include autism spectrum disorders, Rett Syndrome, Tourette Syndrome, Obsessive Compulsive Disorder, insomnia, parasomnias, oppositional defiant disorder, conduct disorder, disruptive mood dysregulation disorder, agitation, anxiety, generalized anxiety disorder, social anxiety disorder, panic disorder, anxiety or agitation due to Alzheimer's dementia, schizophrenia, substance withdrawal syndrome (alcohol, benzodiazepine, barbiturate, and cocaine), post-traumatic stress disorder (PTSD), tremors, essential tremor, spasticity due to cerebral palsy, depression, (including major depression, major depressive disorder, severe depression, unipolar depression, unipolar disorder, or recurrent depression), postnatal or postpartum depression, atypical depression, melancholic depression, Psychotic Major Depression (PMD), catatonic depression, Seasonal Affective Disorder (SAD), dysthymia, double depression, Depressive Personality Disorder (DPD), Recurrent Brief Depression (RBD), minor depressive disorder, bipolar disorder or manic depressive disorder, post-traumatic stress disorders, post-menopausal depression, depression caused by chronic medical conditions, treatment-resistant depression, refractory depression, suicidality, suicidal ideation, mood disorder or suicidal behavior, ganaxolone used as a sedative agent, ganaxolone used as an analgesic agent, attention deficit hyperactivity disorder, or Disruptions in neurocognition including delirium.

The disclosure further includes methods of treating seizures arising from neurodegenerative disorders. Such neurodegenerative disorders include Parkinson's disease, Alzheimer's disease, Amyotrophic Lateral Sclerosis, and Huntington's disease. The disclosure includes methods of treating seizure arising from inflammatory disorders, such as multiple sclerosis. The disclosure includes methods of treating seizure disorders arising from lysosomal storage disorders including Neimann-Pick-C, Tay Sachs, Batten, Sandhoff, and Gaucher disease.

Although preferred subjects are human, typically any mammal including domestic animals such as dogs, cats and horses, may also be treated according to the methods disclosed herein.

The formulations disclosed herein containing an amorphous neurosteroid, preferably ganaxolone dispersed in a polymer complex, surprisingly and unexpectedly have improved bioavailability properties. Accordingly, a lower drug load of ganaxolone can be administered to the patient than would typically be required and still achieve a therapeutic effect, which is beneficial for the subject under treatment.

Due to the improved bioavailability of the formulation the amount administered to the subject may be less than would otherwise be administered using the oral ganaxolone nanosuspension formulation currently used in clinical trials. The amount of the formulation administered can be reduced by about 1X, 2X, 3X, 4X, 5X, 6X, 7X, 8X, 9X, 10X, 11X, 12X, 13X, 14X, 15X, 16X, 17X, 18X, 19X, 20X or greater relative, for example, to the oral ganaxolone nanosuspension formulation. In particular, the amount of the formulation administered can be reduced by at least about 6X relative to the oral ganaxolone nanosuspension formulation.

The amount of the formulation (e.g., amorphous ganaxolone dispersed in a polymer matrix) can be reduced by at least about 5%, about 10%, about 15%, about 20%, about 30%, about 40%, about 50%, about 60%, about 70%, about 80%, about 90% or more relative to the oral ganaxolone nanosuspension formulation.

Dosages of the solid dispersion formulations disclosed herein may be administered in amounts of about 10 mg to 25 mg/day, 25 mg to 50 mg/day, 50 mg to 75 mg/day, 75 mg to 100 mg/day, 100 mg to 125 mg/day, 125 mg to 150 mg/day, 150 mg to 175 mg/day, 175 mg to 200 mg/day, 200 mg to 225 mg/day, 225 mg to 250 mg/day, 250 mg to 275 mg/day, 275 mg to 300 mg/day, 300 mg to 325 mg/day, 325 mg to 350 mg/day, 350 mg to 375 mg/day, 375 mg to 400 mg/day, 400 mg to 425 mg/day, 425 mg to 450 mg/day, 450 mg to 475 mg/day, 475 mg to 500 mg/day, 500 mg to 525 mg/day, 525 mg to 550 mg/day, 550 mg to 575 mg/day, 575 mg to 600 mg/day, 600 mg to 625 mg/day, 625 mg to 650 mg/day, 650 mg to 675 mg/day, 675 mg to 700 mg/day, 700 mg to 725 mg/day, 725 mg to 750 mg/day, 750 mg to 775 mg/day, 775 mg to 800 mg/day, 800 mg to 825 mg/day, 825 mg to 850 mg/day, 850 mg to 875 mg/day, 875 mg to 900 mg/day, 900 mg to 925 mg/day, 925 mg to 950 mg/day, 950 mg to 975 mg/day, 975 mg to 1000 mg/day, 1000 mg to 1025 mg/day, 1025 mg to 1050 mg/day, 1050 mg to 1075 mg/day, 1075 mg to 1100 mg/day, 1100 mg to 1125 mg/day, 1125 mg to 1150 mg/day, 1150 mg to 1175 mg/day, 1175 mg to 1200 mg/day, 1200 mg to 1225 mg/day, 1225 mg to 1250 mg/day, 1250 mg to 1275 mg/day, 1275 mg to 1300 mg/day, 1300 mg to 1325 mg/day, 1325 mg to 1350 mg/day, 1350 mg to 1375 mg/day, 1375 mg to 1400 mg/day, 1400 mg to 1425 mg/day, 1425 mg to 1450 mg/day, 1450 mg to 1475 mg/day, 1475 mg to 1500 mg/day, 1500 mg to 1525 mg/day, 1525 mg to 1550 mg/day, 1550 mg to 1575 mg/day, 1575 mg to 1600 mg/day, 1600 mg to 1625 mg/day, 1625 mg to 1650 mg/day, 1650 mg to 1675 mg/day, 1675 mg to 1700 mg/day, 1700 mg to 1725 mg/day, 1725 mg to 1750 mg/day, 1750 mg to 1775 mg/day, 1775 mg to 1800 mg/day, 1800 mg to 1825 mg/day, 1825 mg to 1850 mg/day, 1850 mg to 1875 mg/day, 1875 mg to 1900 mg/day, 1900 mg to 1925 mg/day, 1925 mg to 1950 mg/day, 1950 mg to 1975 mg/day, or 1975 mg to 2000 mg/day.

These doses may be a safe dose for repeated administration, such as once hourly dosing to once daily dosing, twice daily dosing, dosing one to 12 times daily. In some embodiments, the solid pharmaceutical formulations disclosed herein may be administered once, twice, three times, four times, five times, six times, seven times, eight times, nine times, ten times, eleven times, twelve times, thirteen times, fourteen times, fifteen times or more per day. It is preferable that the solid pharmaceutical formulation disclosed herein is administered between once per day to three times per day.

F. Combination Therapy

The formulations and methods described herein may also be used in conjunction with other therapeutic reagents that are selected for their particular usefulness against the condition that is being treated. In general, the formulations disclosed herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of compounds used will depend upon the diagnosis of the attending physicians and their judgment of the condition of the patient and the appropriate treatment protocol. The compounds may be administered concurrently (e.g., simultaneously, essentially simultaneously or within the same treatment protocol) or sequentially, depending upon the nature of the proliferative disease, the condition of the patient, and the actual choice of compounds used. The determination of the order of administration, and the number of repetitions of administration of each therapeutic agent during a treatment protocol, is well within the knowledge of the skilled physician after evaluation of the disease being treated and the condition of the patient.

It is understood that the dosage regimen to treat, prevent, or ameliorate the condition(s) for which relief is sought, can be modified in accordance with a variety of factors. These factors include the disorder from which the subject suffers, as well as the age, weight, sex, diet, and medical condition of the subject. Thus, the dosage regimen actually employed can vary widely and therefore can deviate from the dosage regimens set forth herein.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several days depending upon the properties of each pharmaceutical agent, such as potency, solubility, bioavailability, plasma half-life and kinetic profile of the pharmaceutical agent. Circadian variation of the target molecule concentration may also determine the optimal dose interval.

In embodiments, the formulations disclosed herein comprising a neurosteroid (e.g., ganaxolone) and a cyclodextrin (e.g., hydroxypropyl-beta-cyclodextrin) is administered with at least one other anti-convulsant agent, anti-epileptic agent, antianxiety agent, or an anti-depression agent.

G. Definitions

All publications and patents cited in this disclosure are incorporated by reference in their entirety. To the extent, the material incorporated by reference contradicts or is inconsistent with this specification, the specification will supersede any such material. The citation of any references herein is not an admission that such references are prior art to the present disclosure. Various terms relating to aspects of the description are used throughout the specification and claims. Such terms are to be given their ordinary meaning in the art unless otherwise indicated. Other specifically defined terms are to be construed in a manner consistent with the definitions provided herein.

The term “amorphous” as used herein indicates that a compound in its solid state lacks significant crystallinity when analyzed via powder X-ray diffraction (XRD).

The term “effective amount” as used herein refers to an amount of a compound or the formulation of the compound described herein (e.g., a neurosteroid, such as ganaxolone), which is sufficient to achieve the desired results under the conditions of administration. For example, an effective amount of a compound or the formulation of the compound described herein for the treatment of an epilepsy disorder is an amount that can manage seizure activity, suppress seizure, allow the patient to recover from a hyperexcitable state, prevents seizure-relapse, or can provide continued suppression of seizure. A skilled clinician can determine appropriate dosing based on a variety of considerations including the severity of the disorder, the subject's age, weight, general health and other consideration.

As used herein, the terms “treat,” treatment,” or “treating” and grammatically related terms, refer to an improvement of any sign, symptoms, or consequence of the disease, such as prolonged survival, less morbidity, and/or a lessening of side effects. As is readily appreciated in the art, full eradication of disease is preferred but not a requirement for treatment.

The term “pharmaceutical compositions” as used herein are compositions comprising at least one active agent, such as a compound or salt, solvate, or hydrate of a neurosteroid, and at least one other substance, such as an excipient. Pharmaceutical compositions optionally contain one or more additional active agents. When specified, pharmaceutical compositions meet the U.S. FDA's GMP (good manufacturing practice) standards for human or non-human drugs. “Pharmaceutical combinations” are combinations of at least two active agents which may be combined in a single dosage form or provided together in separate dosage forms with instructions that the active agents are to be used together to treat a disorder, such as status epilepticus.

The term “subject” as used herein refers to any animal, such as any mammal, including but not limited to, humans, non-human primates, rodents, and the like. In some embodiments, the mammal is a mouse. In some embodiments, the mammal is a human.

The terms “AUC” and “Cmax,” are term of art used in herein in accordance with their normal meaning to refer to pharmacokinetic parameters that may be used to characterize the pharmacokinetic responses of a particular drug product in an animal or human subject. The term “AUC” refers to the “area under the curve” that represents changes in blood, serum, or plasma concentrations of a substance, e.g., ganaxolone, over time. As used herein, the term “Cmax” refers to the maximum or peak blood, serum, or plasma concentration of substance (e.g., ganaxolone) observed in a subject after its administration.

### 6. EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compounds and methods in detail, the same will be more clearly understood by reference to the following examples, which are introduced for illustration only and not intended to be limiting.

### 7. EXAMPLES

### Example 1. Preparation of Amorphous Solid Dispersion Ganaxolone Formulation

The formulation was initially prepared at laboratory scale using a Haake Minilab twin screw compounder. For the material that was evaluated in the rat study, a mixture of 30 parts Ganaxolone to 70 parts of Kollidon VA64 (HME-1) was first blended using a mortar and pestle for 10 minutes. This material was then extruded using the Haake Minilab at 195 deg. C, using a screw speed of 100 rpm and 2-3 minutes of recirculation time (recycle mode).

The same formulation was prepared again at a slightly larger scale for the rat PK study using a Leistritz Nano-16 extruder with the temperature in the first section of the barrel at 185 deg C, the second (middle) section at 190 deg C, and the third section at 195 deg C, just prior to extrusion through a 2.5 mm die kept at 195 deg C. The feed rate was 2 g/min and the rotational speed of the screws was 150 rpm.

The collected extrudate was allowed to cool and then was ground using a small table top Magic Bullet with cross blades grinder. The ground material was sieved through a 100 mesh screen to separate out the larger particles and the fraction which passed through the sieve was used for the study.

A similar process was used for the various compositions that were evaluated including:
30% ganaxolone 50% Kollidon VA64 20% Soluplus by weight (HME-2);
30% ganaxolone 70% Kollidon VA64 (HME-1) by weight;
20% ganaxolone 56% Kollidon VA64 24% Soluplus by weight; and
20% ganaxolone 80% Kollidon VA64 by weight.

### Example 2: Polymer/surfactant screening activity—DSC and micro-tube evaporation

A total of 34 DSC miscibility as well as 20 micro-evap experiments on polymer-surfactant-ganaxolone combinations were conducted to assess the miscibility (DSC) and in-vitro solubility (micro-evap) enhancement of these physical combinations.

Polymers screened were Kollidon VA64 (polyvinyl pyrrohdone-co-vinyl acetate)), HPMCAS-M (hypromellose acetate succinate), HPMCAS-L (hypromellose acetate succinate), HPMCAS-H (hypromellose acetate succinate), PVP K30 (polyvinylpyrrolidone), Soluplus (polyvinylcaprolactamn-poly iny acetate-polycehy lene glycol graft copolymerl, Eudragit S100, Eudragit L100, Eudragit L100-55 and Eudragit EPO. Surfactant/solubilizers screened included sodium lauryl sulfate (SLS), TPGS(D-alpha-tocopherol polyethylene glycol 1000 succinate), PEG1500 (polyethylene glycol 1500), Poloxamer 188 (poly(ethylene glycol)-block-poly(propylene glycol)-block-poly(ethylene glycol), Valproic acid.

The results of DSC and microevaporative screening studies showed that all grades of the HPMCAS as well as Kollidon VA64 showed partial residual crystallinity of ganaxoline in the DSC screening experiments. It is expected that residual crystallinity could result in long-term physical instability. Eudragit S100, L100 and L100-55 showed amorphization of ganaxolone, while Eudragit EPO showed residual partial crystallinity. Soluplus at 80:20 polymer:ganaxolone ratio showed amorphous ganaxolone while at higher drug loads it showed crystalline ganaxolone. Addition of Valproic acid resulted in increased crystallinity of the ganaxolone in all formulations. In microevap experiments, HPMCAS and Eudragit-L100 provided highest ganaxolone supersaturation.

### Example 3: Rat PK Study

A rat pK study was conducted on solid amorphous formulations of ganaxolone. Male Sprague Dawley rats (six groups of six rats per group, age 7-9 weeks) were used in the study. Ganaxolone formulations that contained the ganaxolone from the same solid amorphous formulation batch were prepared and administered per orally according to the schedule in Table 1.

TABLE 1
Dosing schedule

| | | Ganaxolone dose |
|---|---|---|
| | | administered |
| Group | Formulation ganaxolone concentration | (mg/kg) |
| 1 | 1 mg/ml ganaxolone suspension in water | 10 |
| 2 | 2 mg/ml ganaxolone suspension in water | 20 |
| 3 | 1 mg/ml ganaxolone - HME-1 suspension | 10 |
| | in 0.5 wt % HPMC | |
| 4 | 2 mg/ml ganaxolone - HME-1 suspension | 20 |
| | in 0.5 wt % HPMC | |
| 5 | 1 mg/ml ganaxolone - HME-2 suspension | 10 |
| | in 0.5 wt % HPMC | |
| 6 | 2 mg/ml ganaxolone - HME-2 suspension | 20 |
| | in 0.5 wt % HPMC | |

Blood samples (approximately 250 to 300 microliters) were obtained 15 minutes, 30 minutes, 1 hour, 2 hours, 3 hours, 4 hours, 8 hours and 24 hours following administration of the ganaxolone formulations. Blood samples were temporarily held on ice. Then plasma was prepared and stored at −20° C. for up to 24 hours, and then at −80° C.

Plasma was analyzed for ganaxolone concentration by LC/MS using an AB Sciex 5500+instrument, Analyst Software 1.7.1, with Ionization Mode: APCI, positive ions; Scan Mode: Multiple reaction monitoring (MRM), and Ganaxolone-D6 as an internal standard. LC was performed using Kinetex C18 2.6 micron (50 mm* 2.1 mm) column; the mobile phase A was water (with 0.05% formic acid), mobile phase B was MeOH (with 0.05% formic acid).

Plasma samples (50 microliters) were prepared by adding 5 microliters MeOH, then 100 microliters of 100 ng/ml Ganaxolone D6 in MeOH/CAN (v/v 1:1) were added and mixed well. To this was added 1 ml hexane in 1.5 ml tubes and vortexed for 15 minutes, then centrifuges at 13000 rpm for 10 minutes. The tubes were then placed in a dry ice bath for 3 minutes and the bottom water layer was frozen. 800 microliters of the supernatant was transferred into a 96 well-plate (0.65 mL/well) and evaporated under nitrogen until dry at 35° C. Then 200 microliters of 50% MeOH with 01% formic acid was added to each well and mixed well. The sample matrix and the blank matrix were Male SD rat plasma (K₂EDTA as anti-coagulant) No additional peak was observed in the MRM channel as the compound Ganaxolone in plasma samples.

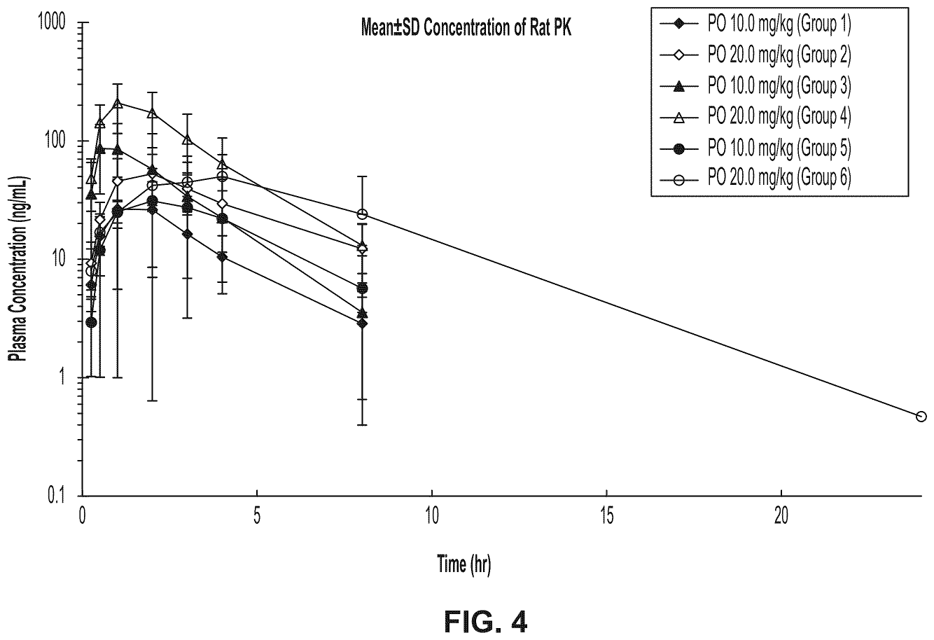

The results are shown in Tables 2-13 and FIG. 4.

TABLE 2
PK Data from Group 1 administered with 10 mg/kg of amorphous ganaxolone

| | | | | | | | | | | | Last | | |
|---|---|---|---|---|---|---|---|---|---|---|---|---|---|
| | | | | | | | | | AUC_{Inf}/ | | time | Time | |
| | No. of | | | | | | | | D | | point | points | |
| | pts | | | | AUCₗₐₛₜ | AUC_{Inf} | AUC | | (hr*kg* | | for | for | |
| | used | t_{1/2} | tₘₐₓ | Cₘₐₓ | (hr*ng/ | (hr*ng/ | Extr | MRT_{Inf} | ng/mL/ | F | AUCₗₐₛₜ | T_{1/2} | |
| Animal | for t_{1/2} | (hr) | (hr) | (ng/mL) | mL) | mL) | (%) | (hr) | mg) | (%) | (hr) | (hr) | Rsq |
| Rat #1 | 3 | 1.12 | 2.00 | 14.0 | 33.4 | 40.0 | 16.5 | 2.45 | 4.00 | NA | 4.00 | 2, 3, | 0.865 |
| | | | | | | | | | | | | 4 | |
| Rat #2 | 3 | 1.87 | 1.00 | 66.2 | 219 | 232 | 5.70 | 3.00 | 23.2 | NA | 8.00 | 3, 4, | 0.992 |
| | | | | | | | | | | | | 8 | |
| Rat #3 | 3 | 2.15 | 1.00 | 32.1 | 92.5 | 99.4 | 6.92 | 2.97 | 9.94 | NA | 8.00 | 3, 4, | 0.983 |
| | | | | | | | | | | | | 8 | |
| Rat #4 | 3 | 3.77 | 1.00 | 23.2 | 88.9 | 117 | 23.9 | 5.61 | 11.7 | NA | 8.00 | 3, 4, | 0.994 |
| | | | | | | | | | | | | 8 | |
| Rat #5 | 3 | 2.65 | 3.00 | 18.4 | 94.8 | 114 | 16.6 | 4.75 | 11.4 | NA | 8.00 | 3, 4, | 0.999 |
| | | | | | | | | | | | | 8 | |
| Rat #6 | 3 | 1.68 | 2.00 | 19.6 | 55.9 | 76.7 | 27.2 | 3.29 | 7.67 | NA | 4.00 | 2, 3, | 0.908 |
| | | | | | | | | | | | | 4 | |
| N | 6 | 6 | 6 | 6 | 6 | 6 | 6 | 6 | 6 | 0 | | | |
| Mean | | 2.21 | 1.67 | 28.9 | 97.4 | 113 | 16.1 | 3.68 | 11.3 | NA | | | |
| SD | | 0.92 | 0.82 | 19.2 | 64.3 | 64.9 | 8.7 | 1.22 | 6.5 | NA | | | |
| CV % | | 41.6 | 49.0 | 66.6 | 66.1 | 57.4 | 53.8 | 33.2 | 57.4 | NA | | | |

TABLE 3
PK Data from Group 2 administered with 20 mg/kg of amorphous ganaxolone

| | | | | | | | | | | | Last | | |
|---|---|---|---|---|---|---|---|---|---|---|---|---|---|
| | | | | | | | | | AUC_{Inf}/ | | time | Time | |
| | No. of | | | | | | | | D | | point | points | |
| | pts | | | | AUCₗₐₛₜ | AUC_{Inf} | AUC | | (hr*kg* | | for | for | |
| | used | t_{1/2} | tₘₐₓ | Cₘₐₓ | (hr*ng/ | (hr*ng/ | Extr | MRT_{Inf} | ng/mL/ | F | AUCₗₐₛₜ | T_{1/2} | |
| Animal | for t_{1/2} | (hr) | (hr) | (ng/mL) | mL) | mL) | (%) | (hr) | mg) | (%) | (hr) | (hr) | Rsq |
| Rat #7 | 3 | 2.68 | 2.00 | 43.9 | 228 | 272 | 16.1 | 4.62 | 13.6 | NA | 8.00 | 3, 4, | 0.998 |
| | | | | | | | | | | | | 8 | |
| Rat #8 | 4 | 1.89 | 2.00 | 50.0 | 184 | 200 | 7.58 | 3.46 | 9.98 | NA | 8.00 | 2, 3, | 0.987 |
| | | | | | | | | | | | | 4, 8 | |
| Rat #9 | 3 | 1.97 | 2.00 | 19.7 | 85.2 | 92.9 | 8.35 | 3.50 | 4.65 | NA | 8.00 | 3, 4, | 0.961 |
| | | | | | | | | | | | | 8 | |
| Rat #10 | 3 | 2.54 | 3.00 | 58.9 | 330 | 390 | 15.4 | 4.75 | 19.5 | NA | 8.00 | 3, 4, | 0.969 |
| | | | | | | | | | | | | 8 | |
| Rat #11 | 3 | 4.58 | 2.00 | 93.4 | 384 | 538 | 28.6 | 6.43 | 26.9 | NA | 8.00 | 3, 4, | 1.00 |
| | | | | | | | | | | | | 8 | |
| Rat #12 | 3 | 5.42 | 2.00 | 59.9 | 197 | 310 | 36.3 | 7.85 | 15.5 | NA | 8.00 | 3, 4, | 0.694 |
| | | | | | | | | | | | | 8 | |
| N | 6 | 6 | 6 | 6 | 6 | 6 | 6 | 6 | 6 | 0 | | | |
| Mean | | 3.18 | 2.17 | 54.3 | 235 | 300 | 18.7 | 5.10 | 15.0 | NA | | | |
| SD | | 1.47 | 0.41 | 24.1 | 108 | 154 | 11.5 | 1.73 | 7.7 | NA | | | |
| CV % | | 46.1 | 18.8 | 44.4 | 45.8 | 51.3 | 61.2 | 33.9 | 51.3 | NA | | | |

TABLE 4
PK Data from Group 3 administered with 10 mg/kg of amorphous ganaxolone

| | | | | | | | | | | | Last | | |
|---|---|---|---|---|---|---|---|---|---|---|---|---|---|
| | | | | | | | | | AUC_{Inf}/ | | time | Time | |
| | No. of | | | | | | | | D | | point | points | |
| | pts | | | | AUCₗₐₛₜ | AUC_{Inf} | AUC | | (hr*kg* | | for | for | |
| | used | t_{1/2} | tₘₐₓ | Cₘₐₓ | (hr*ng/ | (hr*ng/ | Extr | MRT_{Inf} | ng/mL/ | F | AUCₗₐₛₜ | T_{1/2} | |
| Animal | for t_{1/2} | (hr) | (hr) | (ng/mL) | mL) | mL) | (%) | (hr) | mg) | (%) | (hr) | (hr) | Rsq |
| Rat #13 | 3.00 | 1.18 | 0.500 | 100 | 157 | 171 | 8.40 | 1.63 | 17.1 | NA | 4.00 | 2, 3, | 0.947 |
| | | | | | | | | | | | | 4 | |
| Rat #14 | 3.00 | 1.48 | 0.500 | 158 | 660 | 683 | 3.41 | 2.84 | 68.3 | NA | 8.00 | 3, 4, | 0.998 |
| | | | | | | | | | | | | 8 | |
| Rat #15 | 3.00 | 2.84 | 0.500 | 36.8 | 92.8 | 105 | 11.3 | 3.44 | 10.5 | NA | 8.00 | 3, 4, | 0.999 |
| | | | | | | | | | | | | 8 | |
| Rat #16 | 3.00 | 1.43 | 0.500 | 19.0 | 40.1 | 48.9 | 18.1 | 2.38 | 4.89 | NA | 4.00 | 2, 3, | 0.891 |
| | | | | | | | | | | | | 4 | |
| Rat #17 | 3.00 | 1.49 | 1.00 | 147 | 385 | 395 | 2.45 | 2.31 | 39.5 | NA | 8.00 | 3, 4, | 0.993 |
| | | | | | | | | | | | | 8 | |
| Rat #18 | 3.00 | 1.94 | 0.500 | 98.2 | 198 | 207 | 4.12 | 2.30 | 20.7 | NA | 8.00 | 3, 4, | 1.00 |
| | | | | | | | | | | | | 8 | |
| N | 6 | 6 | 6 | 6 | 6 | 6 | 6 | 6 | 6 | 0 | | | |
| Mean | | 1.73 | 0.583 | 93.1 | 256 | 268 | 7.96 | 2.48 | 26.8 | NA | | | |

| | | | | | | | | | |
|---|---|---|---|---|---|---|---|---|---|
| SD | 0.66 | 0.204 | 56.3 | 231 | 261 | 7.17 | 0.60 | 23.5 | NA |
| CV % | 38.1 | 35.0 | 60.5 | 90.3 | 97.2 | 90.0 | 24.3 | 87.6 | NA |

TABLE 5
PK Data from Group 4 administered with 20 mg/kg of amorphous ganaxolone

| | | | | | | | | | | | Last | | |
|---|---|---|---|---|---|---|---|---|---|---|---|---|---|
| | | | | | | | | | AUCInf/ | | time | Time | |
| | No. of | | | | | | | | D | | point | points | |
| | pts | | | | AUClast | AUCInf | AUC | | (hr*kg* | | for | for | |
| | used | t1/2 | tmax | Cmax | (hr*ng/ | (hr*ng/ | Extr | MRTInf | ng/mL/ | F | AUClast | T1/2 | |
| Animal | for t1/2 | (hr) | (hr) | (ng/mL) | mL) | mL) | (%) | (hr) | mg) | (%) | (hr) | (hr) | Rsq |
| Rat | 3.00 | 1.86 | 1.00 | 322 | 1007 | 1069 | 5.81 | 3.01 | 53.4 | NA | 8.00 | 3, 4, | 0.994 |
| #19 | | | | | | | | | | | | 8 | |
| Rat | 3.00 | 2.11 | 1.00 | 144 | 396 | 424 | 6.53 | 2.90 | 21.2 | NA | 8.00 | 3, 4, | 0.996 |
| #20 | | | | | | | | | | | | 8 | |
| Rat | 3.00 | 1.91 | 1.00 | 156 | 410 | 434 | 5.33 | 2.80 | 21.7 | NA | 8.00 | 3, 4, | 0.999 |
| #21 | | | | | | | | | | | | 8 | |
| Rat | 3.00 | 1.63 | 2.00 | 181 | 681 | 712 | 4.29 | 3.04 | 35.6 | NA | 8.00 | 3, 4, | 0.989 |
| #22 | | | | | | | | | | | | 8 | |
| Rat | 3.00 | 1.48 | 1.00 | 335 | 1205 | 1246 | 3.30 | 2.77 | 62.3 | NA | 8.00 | 3, 4, | 1.00 |
| #23 | | | | | | | | | | | | 8 | |
| Rat | 3.00 | 1.68 | 0.500 | 137 | 401 | 415 | 3.34 | 2.33 | 20.8 | NA | 8.00 | 3, 4, | 0.913 |
| #24 | | | | | | | | | | | | 8 | |
| N | 6 | 6 | 6 | 6 | 6 | 6 | 6 | 6 | 6 | 0 | | | |
| Mean | | 1.78 | 1.08 | 213 | 683 | 717 | 4.77 | 2.81 | 35.8 | NA | | | |
| SD | | 0.25 | 0.49 | 91.2 | 350 | 372 | 1.46 | 0.26 | 18.2 | NA | | | |
| CV % | | 13.9 | 45.4 | 42.9 | 51.2 | 51.9 | 30.6 | 9.24 | 50.7 | NA | | | |

TABLE 6
PK Data from Group 5 administered with 10 mg/kg of amorphous ganaxolone

| | | | | | | | | | | | Last | | |
|---|---|---|---|---|---|---|---|---|---|---|---|---|---|
| | | | | | | | | | AUC_{Inf}/ | | time | Time | |
| | No. of | | | | | | | | D | | point | points | |
| | pts | | | | AUCₗₐₛₜ | AUC_{Inf} | AUC | | (hr*kg* | | for | for | |
| | used | t_{1/2} | tₘₐₓ | Cₘₐₓ | (hr*ng/ | (hr*ng/ | Extr | MRT_{Inf} | ng/mL/ | F | AUCₗₐₛₜ | T_{1/2} | |
| Animal | for t_{1/2} | (hr) | (hr) | (ng/mL) | mL) | mL) | (%) | (hr) | mg) | (%) | (hr) | (hr) | Rsq |
| Rat | 5.00 | 4.86 | 1.00 | 11.0 | 40.1 | 65.9 | 39.2 | 7.94 | 6.59 | NA | 8.00 | 1, 2, | 0.562 |
| #25 | | | | | | | | | | | | 3, 4, 8 | |
| Rat | 4.00 | 4.61 | 2.00 | 44.3 | 174 | 272 | 36.2 | 7.56 | 27.2 | NA | 8.00 | 2, 3, | 0.721 |
| #26 | | | | | | | | | | | | 4, 8 | |
| Rat | 4.00 | 2.36 | 2.00 | 38.8 | 193 | 216 | 10.9 | 4.20 | 21.6 | NA | 8.00 | 2, 3, | 0.930 |
| #27 | | | | | | | | | | | | 4, 8 | |
| Rat | 3.00 | 1.29 | 3.00 | 72.5 | 316 | 325 | 2.81 | 3.02 | 32.5 | NA | 8.00 | 3, 4, 8 | 1.00 |
| #28 | | | | | | | | | | | | | |
| Rat | 2.00 | 1.31 | 4.00 | 30.8 | 137 | 144 | 4.84 | 3.86 | 14.4 | NA | 8.00 | 4, 8 | 1.00 |
| #29 | | | | | | | | | | | | | |
| Rat | 2.00 | 0.71 | 3.00 | 10.3 | 28.4 | 32.4 | 12.1 | 2.54 | 3.24 | NA | 4.00 | 3, 4 | 1.00 |
| #30 | | | | | | | | | | | | | |
| N | 6 | 6 | 6 | 6 | 6 | 6 | 6 | 6 | 6 | 0 | | | |
| Mean | | 2.52 | 2.50 | 34.6 | 148 | 176 | 17.7 | 4.85 | 17.6 | NA | | | |
| SD | | 1.74 | 1.05 | 23.3 | 107 | 103 | 19.6 | 2.32 | 11.6 | NA | | | |
| CV % | 69.2 | 42.0 | 67.3 | 72.1 | 58.3 | 110.9 | 47.8 | 65.7 | NA | | | | |

TABLE 7
PK Data from Group 6 administered with 20 mg/kg of amorphous ganaxolone

| | | | | | | | | | | | Last | | |
|---|---|---|---|---|---|---|---|---|---|---|---|---|---|
| | | | | | | | | | AUC_{Inf}/ | | time | Time | |
| | No. of | | | | | | | | D | | point | points | |
| | pts | | | | AUCₗₐₛₜ | AUC_{Inf} | AUC | | (hr*kg* | | for | for | |
| | used | t_{1/2} | tₘₐₓ | Cₘₐₓ | (hr*ng/ | (hr*ng/ | Extr | MRT_{Inf} | ng/mL/ | F | AUCₗₐₛₜ | T_{1/2} | |
| Animal | for t_{1/2} | (hr) | (hr) | (ng/mL) | mL) | mL) | (%) | (hr) | mg) | (%) | (hr) | (hr) | Rsq |
| Rat | 3.00 | 3.98 | 4.00 | 72.5 | 1035 | 1051 | 1.54 | 7.36 | 52.6 | NA | 24.00 | 4, 8, | 0.965 |
| #31 | | | | | | | | | | | | 24 | |
| Rat | 2.00 | 2.83 | 4.00 | 78.1 | 396 | 515 | 23.2 | 5.86 | 25.8 | NA | 8.00 | 4, 8 | 1.00 |
| #32 | | | | | | | | | | | | | |
| Rat | 3.00 | 4.24 | 1.00 | 19.3 | 110 | 159 | 30.5 | 6.76 | 7.95 | NA | 8.00 | 3, 4, 8 | 0.991 |
| #33 | | | | | | | | | | | | | |
| Rat | 2.00 | 3.48 | 4.00 | 59.5 | 325 | 459 | 29.3 | 6.62 | 23.0 | NA | 8.00 | 4, 8 | 1.00 |
| #34 | | | | | | | | | | | | | |
| Rat | 3.00 | 1.92 | 2.00 | 29.6 | 136 | 147 | 7.40 | 3.50 | 7.34 | NA | 8.00 | 3, 4, 8 | 0.994 |
| #35 | | | | | | | | | | | | | |
| Rat | 3.00 | 1.23 | 2.00 | 62.8 | 313 | 321 | 2.41 | 3.26 | 16.0 | NA | 8.00 | 3, 4, 8 | 0.978 |
| #36 | | | | | | | | | | | | | |
| N | 6 | 6 | 6 | 6 | 6 | 6 | 6 | 6 | 6 | 0 | | | |
| Mean | | 2.95 | 2.83 | 53.6 | 386 | 442 | 15.7 | 5.56 | 22.1 | NA | | | |
| SD | | 0.93 | 1.33 | 23.8 | 337 | 368 | 13.0 | 1.76 | 16.7 | NA | | | |
| CV | | 31.7 | 46.9 | 44.4 | 87.4 | 83.2 | 82.9 | 31.6 | 75.6 | NA | | | |
| % | | | | | | | | | | | | | |

TABLE 8
Plasma exposure in Group 1 administered with 10 mg/kg of amorphous ganaxolone
PO 10.0 mg/kg (Group 1)

| | Calculated Concentration (ng/mL) | | | | | | | | |
|---|---|---|---|---|---|---|---|---|---|
| Time (h) | Rat #1 | Rat #2 | Rat #3 | Rat #4 | Rat #5 | Rat #6 | Mean | SD | CV(%) |
| 0.25 | 4.33 | 9.69 | 10.2 | 2.74 | 4.25 | 5.32 | 6.09 | 3.10 | 51.0 |
| 0.5 | 8.56 | 31.1 | 19.2 | 10.8 | 13.6 | 10.6 | 15.6 | 8.4 | 53.8 |
| 1 | 10.8 | 66.2 | 32.1 | 23.2 | 13.2 | 13.8 | 26.6 | 21.0 | 79.1 |
| 2 | 14.0 | 64.4 | 24.8 | 17.8 | 16.2 | 19.6 | 26.1 | 19.1 | 73.1 |
| 3 | 4.95 | 33.0 | 12.0 | 13.2 | 18.4 | 16.3 | 16.3 | 9.4 | 57.5 |
| 4 | 4.07 | 19.2 | 6.97 | 10.2 | 13.7 | 8.60 | 10.5 | 5.4 | 51.2 |
| 8 | BLOQ | 4.90 | 2.22 | 5.14 | 4.93 | BLOQ | 2.87 | 2.47 | 86.1 |
| 24 | BLOQ | BLOQ | BLOQ | BLOQ | BLOQ | BLOQ | NA | NA | NA |

TABLE 9
Plasma exposure in Group 2 administered with 20 mg/kg of amorphous ganaxolone
PO 20.0 mg/kg (Group 2)

| Time | Calculated Concentration (ng/mL) | | | | | | | | |
|---|---|---|---|---|---|---|---|---|---|
| (h) | Rat #7 | Rat #8 | Rat #9 | Rat #10 | Rat #11 | Rat #12 | Mean | SD | CV(%) |
| 0.25 | 9.85 | 6.43 | 5.39 | 12.4 | 16.8 | 4.79 | 9.28 | 4.69 | 50.6 |
| 0.5 | 22.5 | 18.5 | 12.1 | 18.1 | 37.9 | 20.2 | 21.6 | 8.72 | 40.5 |
| 1 | 43.2 | 38.9 | 19.2 | 47.1 | 93.0 | 31.4 | 45.5 | 25.3 | 55.6 |
| 2 | 43.9 | 50.0 | 19.7 | 49.0 | 93.4 | 59.9 | 52.7 | 24.1 | 45.7 |
| 3 | 42.1 | 36.4 | 17.9 | 58.9 | 49.8 | 31.6 | 39.5 | 14.3 | 36.3 |
| 4 | 30.5 | 19.70 | 8.73 | 57.7 | 42.5 | 17.9 | 29.5 | 18.0 | 61.1 |
| 8 | 11.3 | 5.55 | 2.73 | 16.4 | 23.3 | 14.4 | 12.3 | 7.5 | 61.0 |
| 24 | BLOQ | BLOQ | BLOQ | BLOQ | BLOQ | BLOQ | NA | NA | NA |

TABLE 10
Plasma exposure in Group 3 administered with 10 mg/kg of amorphous ganaxolone
PO 10.0 mg/kg (Group 3)

| | Calculated Concentration (ng/mL) | | | | | | | | |
|---|---|---|---|---|---|---|---|---|---|
| Time | Rat | Rat | Rat | Rat | Rat | Rat | | | |
| (h) | #13 | #14 | #15 | #16 | #17 | #18 | Mean | SD | CV(%) |
| 0.25 | 23.5 | 94.2 | 20.8 | 9.65 | 33.8 | 31.0 | 35.5 | 30.0 | 84.5 |
| 0.5 | 99.6 | 158 | 36.8 | 19.0 | 106 | 98.2 | 86.3 | 50.6 | 58.7 |
| 1 | 93.2 | 141 | 31.0 | 15.7 | 147 | 82.6 | 85.1 | 54.4 | 63.9 |
| 2 | 27.5 | 151 | 15.3 | 11.4 | 105 | 35.9 | 57.7 | 57.0 | 98.9 |
| 3 | 12.0 | 109 | 9.65 | 5.22 | 49.4 | 18.2 | 33.9 | 40.0 | 118 |
| 4 | 8.47 | 75.8 | 7.93 | 4.31 | 25.4 | 12.6 | 22.4 | 27.2 | 121 |
| 8 | BLOQ | 10.9 | 2.89 | BLOQ | 4.50 | 3.04 | 3.56 | 4.02 | 113 |
| 24 | BLOQ | BLOQ | BLOQ | BLOQ | BLOQ | BLOQ | NA | NA | NA |

TABLE 11
Plasma exposure in Group 4 administered with 20 mg/kg of amorphous ganaxolone
PO 20.0 mg/kg (Group 4)

| | Calculated Concentration (ng/mL) | | | | | | | | |
|---|---|---|---|---|---|---|---|---|---|
| Time | Rat | Rat | Rat | Rat | Rat | Rat | | | |
| (h) | #19 | #20 | #21 | #22 | #23 | #24 | Mean | SD | CV(%) |
| 0.25 | 47.9 | 28.6 | 33.3 | 30.6 | 86.3 | 61.5 | 48.0 | 22.6 | 47.0 |
| 0.5 | 173 | 115 | 90.0 | 93.7 | 243 | 137 | 142 | 58 | 41.0 |
| 1 | 322 | 144 | 156 | 163 | 335 | 132 | 209 | 94 | 44.8 |
| 2 | 255 | 91.5 | 96.3 | 181 | 291 | 120 | 172 | 85 | 49.2 |
| 3 | 156 | 49.0 | 52.1 | 101 | 204 | 56.2 | 103 | 64 | 62.5 |
| 4 | 92.4 | 31.6 | 34.5 | 83.3 | 123 | 19.2 | 64.0 | 41.4 | 64.8 |
| 8 | 23.1 | 9.11 | 8.37 | 13.0 | 19.3 | 5.73 | 13.1 | 6.8 | 51.9 |
| 24 | BLOQ | BLOQ | BLOQ | BLOQ | BLOQ | BLOQ | NA | NA | NA |

TABLE 12
Plasma exposure in Group 5 administered with 10 mg/kg of amorphous ganaxolone
PO 10.0 mg/kg (Group 5)

| | Calculated Concentration (ng/mL) | | | | | | | | |
|---|---|---|---|---|---|---|---|---|---|
| Time | Rat | Rat | Rat | Rat | Rat | Rat | | | |
| (h) | #25 | #26 | #27 | #28 | #29 | #30 | Mean | SD | CV(%) |
| 0.25 | 3.07 | 4.74 | 3.46 | 4.87 | BLOQ | 1.44 | 2.93 | 1.91 | 65.0 |
| 0.5 | 4.62 | 12.1 | 17.4 | 31.0 | 3.37 | 3.15 | 11.9 | 10.9 | 91.5 |
| 1 | 11.0 | 29.1 | 23.8 | 71.4 | 8.04 | 8.13 | 25.2 | 24.2 | 96.0 |
| 2 | 8.37 | 44.3 | 38.8 | 65.7 | 21.2 | 8.57 | 31.2 | 22.6 | 72.5 |
| 3 | 3.91 | 23.6 | 30.2 | 72.5 | 23.5 | 10.3 | 27.3 | 24.1 | 88.3 |
| 4 | 3.86 | 19.5 | 33.9 | 40.6 | 30.8 | 3.86 | 22.1 | 15.7 | 71.0 |
| 8 | 3.69 | 14.8 | 6.94 | 4.89 | 3.69 | BLOQ | 5.67 | 5.01 | 88.4 |
| 24 | 41.1 | BLOQ | BLOQ | BLOQ | BLOQ | BLOQ | NA | NA | NA |

TABLE 13
Plasma exposure in Group 6 administered with 20 mg/kg of amorphous ganaxolone
PO 20.0 mg/kg (Group 6)

| | Calculated Concentration (ng/mL) | | | | | | | | |
|---|---|---|---|---|---|---|---|---|---|
| Time | Rat | Rat | Rat | Rat | Rat | Rat | | | |
| (h) | #31 | #32 | #33 | #34 | #35 | #36 | Mean | SD | CV(%) |
| 0.25 | 12.3 | 4.34 | 4.10 | 4.29 | 13.6 | 8.99 | 7.94 | 4.32 | 54.4 |
| 0.5 | 13.1 | 13.7 | 13.2 | 13.8 | 18.9 | 28.2 | 16.8 | 6.0 | 35.6 |
| 1 | 20.8 | 22.4 | 19.3 | 22.9 | 26.5 | 37.3 | 24.9 | 6.6 | 26.4 |
| 2 | 43.3 | 49.3 | 16.4 | 50.2 | 29.6 | 62.8 | 41.9 | 16.5 | 39.4 |
| 3 | 46.7 | 69.7 | 17.5 | 48.9 | 22.7 | 62.8 | 44.7 | 21.0 | 46.9 |
| 4 | 72.5 | 78.1 | 16.1 | 59.5 | 18.2 | 55.2 | 49.9 | 26.7 | 53.5 |
| 8 | 71.8 | 29.3 | 7.93 | 26.8 | 3.92 | 4.35 | 24.0 | 26.0 | 108 |
| 24 | 2.81 | BLOQ | BLOQ | BLOQ | BLOQ | BLOQ | 0.468 | NA | NA |

### Example 4: Phase I Single Ascending Dose Study of Hot Melt Extrusion in Healthy Volunteers

A Phase I single ascending dose study in healthy volunteers was conducted using an amorphous formulation (HME-1) of ganaxolone. Subjects (n=12) were grouped into 6 groups each containing 2 subjects, and were administered ganaxolone suspension or amorphous ganaxolone formulation (HME-1 formulation) at day 1, day 5, day 9, day 13, day 17, and day 21 according to the dosing schedule and treatment plan provided in Table 14.

For clinical dosing, at any dose, the desired quantity of solid amorphous ganaxolone (HME-1) was weighed out and 240 mL water was added to HME-1 prior to administration. The formulation was administered orally to the subject.

TABLE 14
Dosing schedule and treatment plan.

| | Treatment Sequence | | | | | |
|---|---|---|---|---|---|---|
| Sequence | Day 1 | Day 5 | Day 9 | Day 13 | Day 17 | Day 21 |
| A (n = 2) | 1 | 4 | 2 | 3 | 5 | 6 |
| B (n = 2) | 2 | 1 | 5 | 3 | 6 | 4 |
| C (n = 2) | 3 | 2 | 1 | 6 | 4 | 5 |
| D (n = 2) | 7 | 10 | 8 | 9 | 11 | 12 |
| E (n = 2) | 8 | 7 | 11 | 9 | 12 | 10 |
| F (n = 2) | 9 | 8 | 7 | 12 | 10 | 11 |

Treatment 1: A single 400 mg dose of ganaxolone suspension (1×8 mL of the 50 mg/mL oral suspension) administered under fed condition.

Treatment 2: A single 100 mg dose of amorphous ganaxolone formulation (HME-1 formulation) (333 mg of powder in 240 ml of water) administered under fasting conditions.

Treatment 3: A single 100 mg dose of amorphous ganaxolone formulation (HME-1 formulation) (333 mg of powder in 240 ml of water) administered under fed conditions.

Treatment 4: A single 200 mg dose of amorphous ganaxolone formulation (HME-1 formulation) (333 mg of powder in 240 ml of water) administered under fed conditions.

Treatment 5: A single 400 mg dose of amorphous ganaxolone formulation (HME-1 formulation) (333 mg of powder in 240 ml of water) administered under fasting conditions.

Treatment 6: A single 400 mg dose of amorphous ganaxolone formulation (HME-1 formulation) (333 mg of powder in 240 ml of water) administered under fed conditions.

Treatment 7: A single 600 mg dose of ganaxolone suspension (1×12 mg of 50 mg/ml oral suspension) administered under fed conditions.

Treatment 8: A single dose of up to 400 mg of amorphous ganaxolone formulation (HME-1 formulation) (333 mg of powder in 240 ml of water) administered under fasting conditions.

Treatment 9: A single dose of up to 400 mg of amorphous ganaxolone formulation (HME-1 formulation) (333 mg of powder in 240 ml of water) administered under fed conditions.

Treatment 10: A single dose of up to 600 mg of amorphous ganaxolone formulation (HME-1 formulation) (333 mg of powder in 240 ml of water) administered under fed conditions.

Treatment 11: A single dose of up to 900 mg amorphous ganaxolone formulation (HME-1 formulation) (333 mg of powder in 240 ml of water) administered under fasting conditions.

Treatment 12: A single dose of up to 900 mg of amorphous ganaxolone formulation (HME-1 formulation) (333 mg of powder in 240 ml of water) administered under fed conditions.

Ganaxolone plasma concentration and ganaxolone area under curve (AUCₜ.o) were measured.

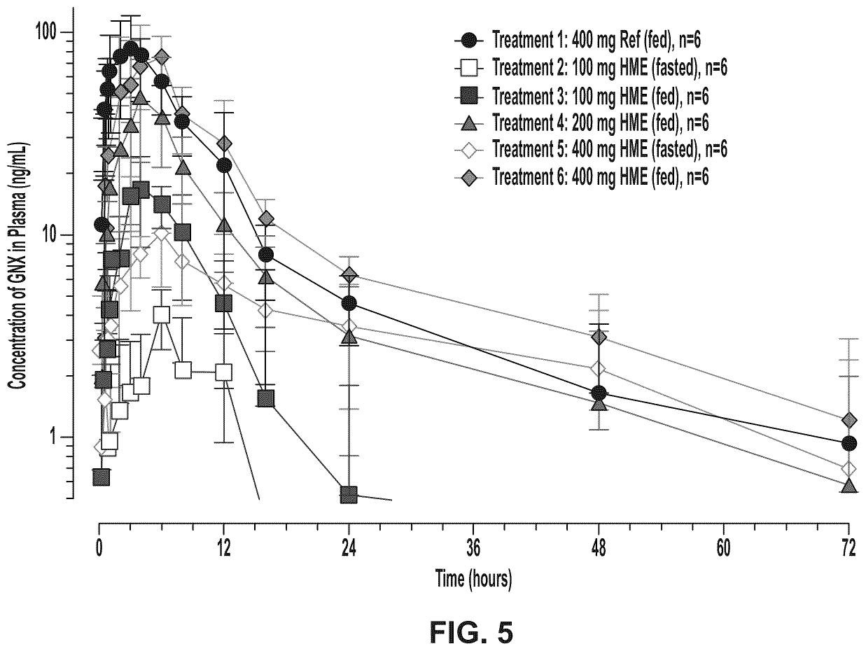

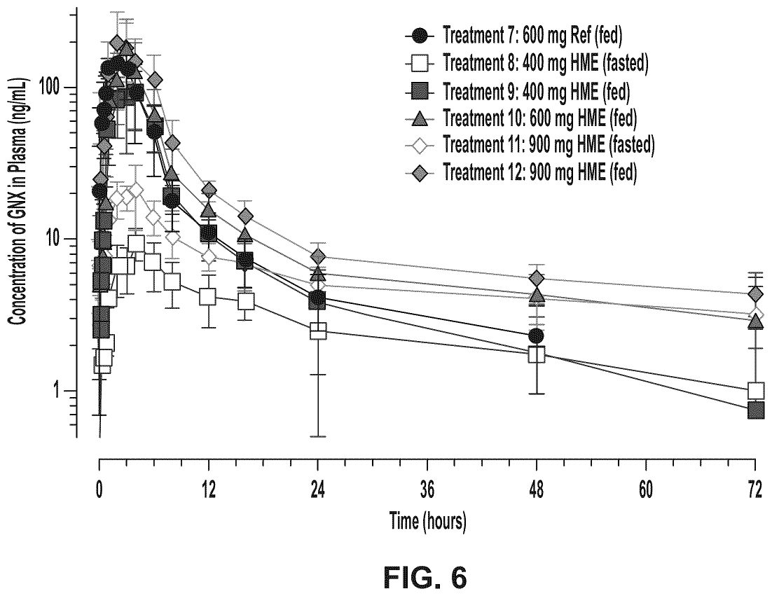

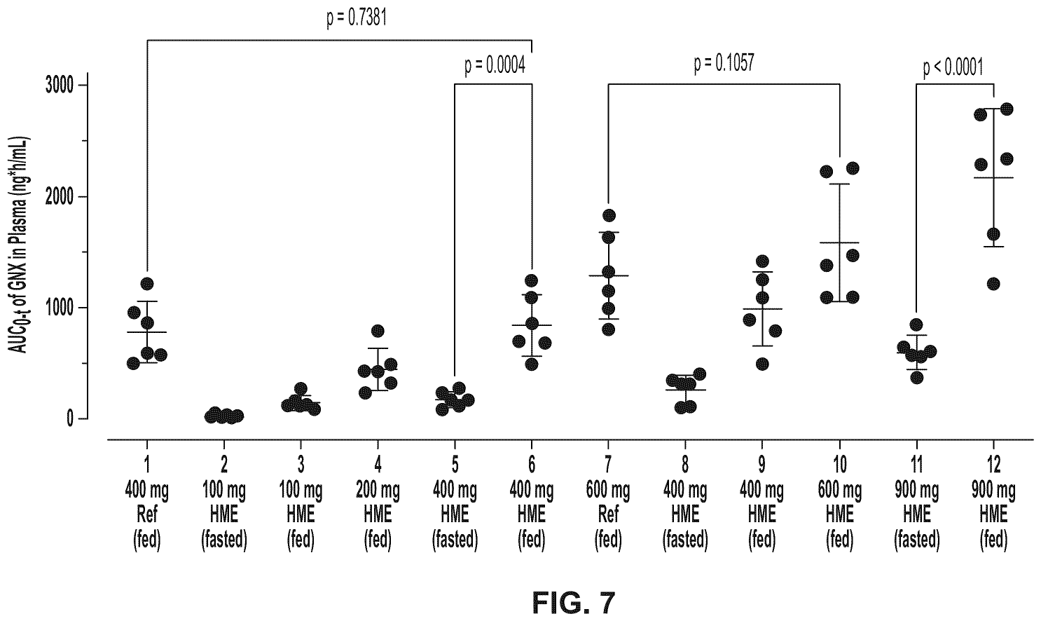

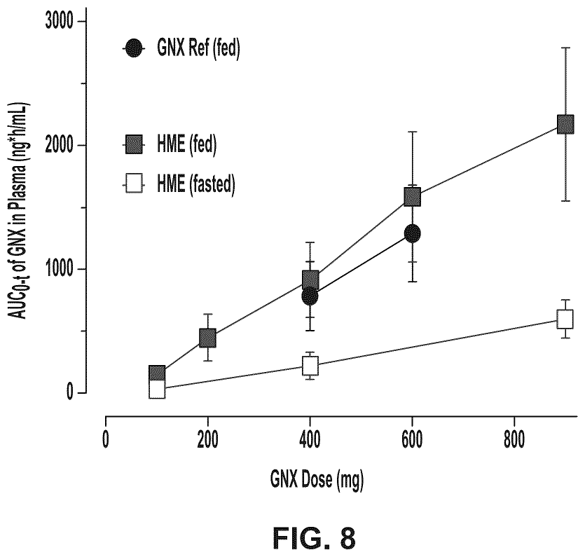

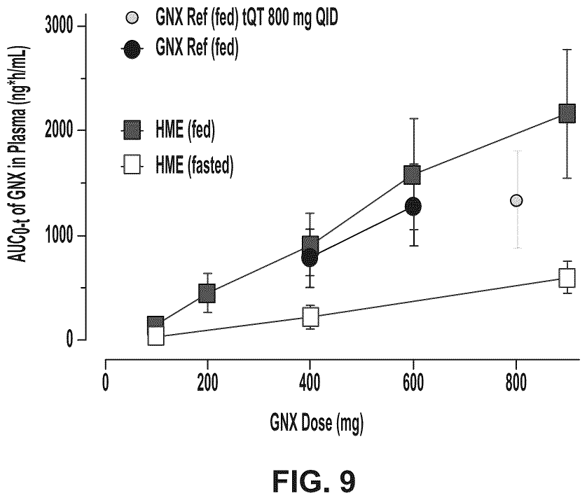

Pharmacokinetic analysis showed that the HME-1 formulation achieves a Cₘₐ that is comparable to ganaxolone suspension. However, at doses of 600 mg or more, HME-1 produced higher exposure (increase in AUC) with little or no change to the Cₘₐ relative to ganaxolone suspension. Results are shown in FIGS. 5 and 6. The results further show that ganaxolone exposure (AUC) increased linearly in proportion to the administered dose of HME-1, including an approximately 37% increase in AUC from 600 mg to 900 mg, without increasing the Cₘₐₓ In contrast, ganaxolone exposure (AUC) following administration of the ganaxolone suspension was not linear with dose and plateaued between 600 mg and 800 mg ganaxolone. Results are shown in FIGS. 7, 8 and 9.

The results of the study demonstrate that higher ganaxolone exposure (AUC) is achieved with the solid amorphous ganaxolone formulation (HME-1 formulation) without significantly increasing peak levels in comparison to the ganaxolone suspension. This pharmacokinetic profile of the HME-1 formulation provides for increase ganaxolone exposure, but without concomitant increase in dose-related (e.g., Cmax-related) adverse effects, as is supportive of twice-daily or less frequent dosing.

The data also demonstrate that administration of HME-1 formulation in a fed state has a large and positive effect on Cₘₐₓ, exposure and ganaxolone area under the curve.

1-56. (canceled)

57. A pharmaceutical composition comprising ganaxolone dispersed in a polymer matrix, wherein
the ganaxolone in present in an amount of up to about 30% by weight;
the polymer matrix comprises Kollidon V64 (BASF, copovidone, copolyvidone, vinylpyrrolidone-vinyl acetate copolymer);
the ganaxolone is substantially amorphous;
the pharmaceutical composition is a solid composition; and wherein all weight percentages are with respect to the pharmaceutical composition.

58. The pharmaceutical composition of claim 57, wherein the ganaxolone is present in an amount of about 10% to about 30% by weight.

59. The pharmaceutical composition of claim 57, wherein ganaxolone is present in an amount of about 30% by weight.

60. The pharmaceutical composition of claim 57, wherein the ganaxolone is present in an amount of about 20% by weight.

61. The pharmaceutical composition of claim 57, wherein the Kollidon V64 (BASF, copovidone, copolyvidone, vinylpyrrolidone-vinyl acetate copolymer) is present in an amount of about 50% to about 80% by weight.

62. The pharmaceutical composition of claim 57, wherein the Kollidon V64 (BASF, copovidone, copolyvidone, vinylpyrrolidone-vinyl acetate copolymer) is present in an amount of about 70% by weight.

63. The pharmaceutical composition of claim 57, wherein the w/w ratio of the Kollidon V64 (BASF, copovidone, copolyvidone, vinylpyrrolidone-vinyl acetate copolymer) to ganaxolone is about 50:30 to about 70:30.

64. The pharmaceutical composition of claim 57, wherein the pharmaceutical composition contains substantially no crystalline ganaxolone.

65. The pharmaceutical composition of claim 57, wherein the crystallinity is measured using X-ray diffraction.

66. The pharmaceutical composition of claim 57, wherein the pharmaceutical composition is in the form of a powder.

67. The pharmaceutical composition of claim 57, wherein the pharmaceutical composition is in a unit dosage form comprising a powder.

68. The pharmaceutical composition of claim 57, comprising a capsule.

69. The pharmaceutical composition of claim 57, comprising a tablet.

70. The pharmaceutical composition of claim 57, wherein the pharmaceutical composition consists essentially of ganaxolone in an amount of about 30% by weight, and Kollidon V64 (BASF, copovidone, copolyvidone, vinylpyrrolidone-vinyl acetate copolymer) in an amount of about 70% by weight.

71. A pharmaceutical composition comprising ganaxolone dispersed in a polymer matrix, wherein
the ganaxolone in present in an amount of up to about 30% by weight;
the polymer matrix comprises Kollidon V64 (BASF, copovidone, copolyvidone, vinylpyrrolidone-vinyl acetate copolymer) in an amount of about 70% by weight;
the ganaxolone is substantially amorphous;
the pharmaceutical composition is a solid composition; and wherein all weight percentages are with respect to the pharmaceutical composition.

72. The pharmaceutical composition of claim 71, wherein the ganaxolone is present in an amount of about 10% to about 30% by weight.

73. The pharmaceutical composition of claim 71, wherein ganaxolone is present in an amount of about 30% by weight.

74. The pharmaceutical composition of claim 71, wherein the pharmaceutical composition contains substantially no crystalline ganaxolone.

75. The pharmaceutical composition of claim 71, wherein the pharmaceutical composition is in the form of a powder.

76. The pharmaceutical composition of claim 71, wherein the pharmaceutical composition is in a unit dosage form comprising a powder.

77. The pharmaceutical composition of claim 71, comprising a capsule.

78. The pharmaceutical composition of claim 71, comprising a tablet.

79. The pharmaceutical composition of claim 71, wherein the pharmaceutical composition consists essentially of ganaxolone in an amount of about 30% by weight, and Kollidon V64 (BASF, copovidone, copolyvidone, vinylpyrrolidone-vinyl acetate copolymer) in an amount of about 70% by weight.
